(19) United States
(12) Patent Application Publication
(10) Pub. No.: US 2003/0113298 A1
(43) Pub. Date: Jun. 19, 2003

(54) Use of bacterial phage associated lysing proteins for the prophylactic and therapeutic treatment of various illnesses

(76) Inventors: Vincent Fischetti, West Hompsteed, NY (US); LAwrance Loomis, Columbia, MD (US); David Trudil, Reisterstown, MD (US)

Correspondence Address: MARVIN A. MOTSENBOCKER, Heller Ehrman White & McAuliffe LLP, Suite 300, 1666 K Street, NW, Washington, DC, 20006, US

(21) Appl. No.: 09/846,688

(22) Filed: May 2, 2001

Related U.S. Application Data
(63) Continuation-in-part of application No. 09/497,495, filed on Apr. 18, 2000, now Pat. No. 6,238,661.
Application No. 09/497,495 is a continuation of application No. 09/395,636, filed on Sep. 14, 1999, now Pat. No. 6,056,954.
Application No. 09/395,636 is a continuation of application No. 08/962,523, filed on Oct. 31, 1997, now Pat. No. 5,997,862.

(51) Int. Cl. A61K 38/48; A61K 39/40

(52) U.S. Cl. 424/93.6; 424/94.63; 424/164.1

(57) ABSTRACT

A composition and method for treating bacterial infections is disclosed which comprises the treatment of an individual with an effective amount of at least one lytic protein, peptide, or peptide fragment thereof, wherein said at least one lytic protein or peptides is in a natural or modified form The composition further discloses several carrier compositions suitable for site-specific delivery of the composition. Further disclosed are the method of use of the composition of the invention. These methods include therapeutic, diagnostic, prognostic and drug screening methods.

This is a continuation-in-part of U.S. patent application Ser. No. 09/497,495, filed Apr. 14, 2000, which is a continuation of U.S. patent application Ser. No. 09/395,636, filed Sep. 14, 1999, now U.S. Pat. No. 6,056,954 which is a continuation of U.S. patent application Ser. No. 08/962,523, filed Oct. 3, 1997, now U.S. Pat. No. 5,997,862.

### BACKGROUND OF THE INVENTION

1. Field of the Invention

The present invention relates to methods and compositions for the treatment of bacterial infections by the use of bacteria-associated phage proteins, or peptides and peptide fragments thereof. More specifically, the invention pertains to phage lytic and/or holin proteins, or peptides and peptide fragments thereof, blended with a carrier for the treatment and prophylaxis of bacterial infection.

2. Description of the Prior Art

Synthetic chemical antibiotics have been used to treat bacterial infections for many years. During this use, their chemical structures have been modified to make them more powerful and in some case, to provide alternate structures that alleviate allergies induced or made worse by their use.

In addition to treating standard sepsis conditions, antibiotics have found a role in the treatment of other, less life threatening conditions such as use of aminopenicillin for acne, as described in U.S. Pat. No. 5,260,292 (Robinson et al.). Methods and compositions for topically treating acne and acneiform dermal disorders include applying an amount of an antibiotic selected from the group consisting of ampicillin, amoxicillin, other aminopenicillins, and cephalosporins, and derivatives and analogs thereof, effective to treat the acne and acneiform dermal disorders. Robinson et al. U.S. Pat. No. 5,409,917 is a representative publication in this area and describes the topical treatment of acne with cephalosporins.

Unfortunately, antibiotics have been used at an ever increasing rate for various illnesses, and this has led to the emergence of antibiotic resistance in bacteria. Larger doses of stronger antibiotics now are used to treat ever more resistant strains of bacteria and multiple antibiotic resistant bacteria have developed. This has prompted longer treatment times with more powerful antibiotics, some of which have deleterious side effects.

Another problem with the treatment of bacterial infections is that most such infections occur at mucosal surface(s) of the body (i.e. a wetted surface such as a cut, or in the mouth, throat, nasal passages, upper respiratory tract, urogenital areas, the eyes and ears). Antibiotics do not easily penetrate many mucosal surfaces such as mucus linings.

Yet another problem is that the number of people allergic to antibiotics appears to be increasing. Having another type of antibiotic would be helpful in broadening the range of tools that a medical practitioner can use for allergic patients.

In view of these problems with standard antibiotics, bacteriophages have been proposed for treating bacterial infections, as for example, described in U.S. Pat. No. 5,688,501 (Merril, et al.), which teaches a treatment method that uses bacteriophage (either lytic or non-lytic) that target specific host bacteria. U.S. Pat. No. 4,957,686 (Norris) discloses a procedure to improve dental hygiene by introducing bacteriophage into the mouth that are parasitic to bacteria that adhere to the salivary pellicle.

Unfortunately the direct use of bacteriophages to prevent or fight disease has disadvantages. Both the phage and the targeted bacteria should be in an optimum phase of their growth cycless. Further, the ratio of phage to bacteria should be optimized. If too many or too few phages are applied, phage may not attach properly and/or suitable amounts of lysing protein may not be produced. Still another limitation is the instability of phages in vivo. Bacteriophages are sensitive to changes in their growing condition and are inhibited by substances within the body, including bacterial debris from phage infected bacteria. Yet another limitation to the use of phage is the possibility of immunological reactions, rendering the phage non-functional.

While studying these these and other problems, Fischetti et al. discovered that phage lytic proteins specific for a bacterium infected with a specific phage can effectively and efficiently break down the cell wall of the bacterium. See for example, U.S. Pat. No. 5,604,109 (Fischetti et al), the contents of which are incorporated by reference in its entirety. Because the lytic and holin proteins of generally lack of proteolytic activity, they are non-destructive to mammalian proteins and are particularly desirable anti-bacterial agents.

The use of the phage associated lytic and/or holin proteins for the prophylactic and therapeutic treatment of bacterial diseases, particularly for mucosal surfaces and with appropriate carriers, however, has not been fully explored.

### SUMMARY OF THE INVENTION

The present invention arose from discoveries pertaining to the bacteriophage associated lytic proteins and holin proteins useful for destroying bacteria, including isozymes, analogs, and variants thereof in a natural or modified form either alone or in combination with complementary agents. The invention also features composition that are site-specific for the mucosal membranes and pharmaceutically acceptable carriers for the treatment and amelioration of the infection of mucus membrane.

Accordingly, in one aspect, the present invention provides a pharmaceutical composition containing at least one bacteria-associated phage protein and peptides and peptide fragments thereof, isolated from one or more bacteria species, which phage proteins and peptides fragments thereof include phage lytic and/or holin proteins. In one embodiment, the lytic and/or holin proteins, including their isozymes, analogs, or variants, are used in a modified form. In another embodiment the lytic and/or holin proteins, including their isozymes, analogs, or variants, are used in a modified form or a combination of natural and modified forms. The modified forms of lytic and holin proteins are made synthetically by chemical synthesis and/or DNA recombinant techniques.

The invention features compositions containing at least one natural lytic protein, including isozymes, analogs, or variants thereof, isolated from the same or a different bacteria, with optional addition of a complementary agent.

According to one embodiment, the pharmaceutical composition includes one or more modified lytic protein, including isozymes, analogs, or variants thereof, produced by chemical synthesis or DNA recombinant techniques. In particular modified lytic protein is produced by chimerization , shuffling, or both. Preferably, the pharmaceutical composition contains combination of one or more natural lytic protein and one or more chimeric or shuffled lytic protein.

According to another embodiment of the invention, the pharmaceutical composition contains a peptide or a peptide fragment of at least one lytic protein derived from the same or different bacteria species, with an optional addition of one or more complementary agent, and a pharmaceutically acceptable carrier.

According to another embodiment of the invention, the pharmaceutical composition contains a peptide or a peptide fragment of at least one holin protein, or at least one holin and one lytic protein, which lytic and holin proteins are each derived from the same or different bacteria species, with an optional addition of a complementary agents, and a suitable carrier or diluent.

Also within the scope of the invention are compositions containing nucleic acid molecules that either alone or in combination with other nucleic acid moleucles are capable of expressing an effective amount of lytic and/or holin proteins or a peptide fragment of the lytic and/or holin proteins in vivo. Also encompassed within the scope of this invention are cell cultures containing these nucleic acid molecules polynucleotides and vectors carrying and expressing these molecules in vitro or in vivo.

According to another embodiment of the invention, the pharmaceutical composition contains a complementary agent, including one or more conventional antibiotics.

According to another aspect of the invention, the pharmaceutical composition contains antibodies directed against a phage protein or peptide fragment of the invention.

According to another aspect, the invention provides, prevention, amelioration, or treatment of a variety of illnesses caused by Gram negative and/or Gram positive bacteri, including Streptococcal pyogenes, Streptococcal pneumoniae, Streptococcus fasciae, Hemophilus influenza, Listeria, Salmonella, E. coli, and Campylobacter.

The bacteria-phage associated proteins of this invention are administered to subjects in need thereof via several means of application. Means of application includes suitable carries that assist in delivery of the composition to the site of the infection and subsequent adsorption of the composition. The composition containing lytic and/or holin proteins or peptides and peptide fragments thereof, are incorporated into pharmaceutically acceptable carries and placed into appropriate means of application. Preferably, application means include suppository enemas, liquid means (for example, syrups, mouthwash, and eye drops in aqueous or non-aqueous form), solid means (for example, food stuff, confectionary, and toothpaste), bandages, tampons, topical creams, and inhalers, among others.

According to an embodiment of the invention, one or more phage proteins, or peptides and peptide fragments thereof, are placed in an inhaler to treat or prevent the spread of diseases localized in the mucus lining of the oral cavity and lungs. In a preferred embodiment, specific lytic proteins for tuberculosis are placed in a carrier and used to prevent or treat tuberculosis. In another embodiment, phage proteins are administered in the form of candy, chewing gum, lozenge, troche, tablet, a powder, aerosol, liquid spray, or toothpaste for the prevention or treatment of bacterial infections associated with upper respiratory tract illnesses.

According to another embodiment of invention, eye drops containing lytic proteins of Hemophilus, Pseudomonas, and/or Staphylococcus are used to directly treat eye infections.

In another embodiment of the invention, specific lytic proteins are used in the treatment of bacterial infections associated with topical or dermatological infections, administered in the form of a topical ointment or cream.

The invention also provides composition and method to treat burns and wounds by using one or more phage proteins, including preferably phage associated with Staphylococcus or Pseudomonas, incorporated into bandages to prevent or treat infections of bums and wounds.

According to another embodiment, lytic proteins, including those proteins or peptides and peptide fragments thereof specific for group B Streptococcus, are incorporated into tampons to prevent infection of the neonate during birth without disturbing normal vaginal flora so that women would not be overcome by yeast infection as a result of antibiotic therapy. Vaginal infections caused by Group B Streptococcus can cause premature birth and subsequent complications resulting in neonatal sepsis.

According to yet another embodiment of the invention, the pharmaceutical composition contains phage polypeptides, peptide fragments, nucleic acid molecules encoding phage protein or protein peptides fragments, antibody and antibody fragments, having biological activity either alone or with combination of other molecules polypeptides, peptides. In particular the phage polypeptides are selected from the group consisting of: a natural phage polypeptide, a naturally occurring allelic variant of said polypeptide, a modified polypeptide, and a polypeptide which is encoded by a nucleic acid molecule comprising a nucleotide sequence which is at least 65% identical to a nucleic acid encoding the said natural peptide. Additionally, the polypeptide of the invention is attached to heterologous amino acid sequences

According to another embodiment of the invention, phage peptides and peptide fragments thereof are antibodies that selectively bind to phage polypeptides.

The invention also features nucleic acid molecules as phage peptides and peptide fragments thereof The nucleic acid molecules of the invention are preferably attached to regulatory sequences and signal sequences, wherein said sequences affect site specificity and trans-membrane movements of said nucleic acid molecules. The signal sequences affect transportation of the nucleic acid molecules to the mucous membranes.

According to another aspect of the invention, a method for detecting the presence of a phage protein or peptides and peptide fragments thereof of the invention in a sample comprises: contacting the sample with a compound which selectively binds to said phage protein or peptides and peptide fragments thereof of claim 1; and determining whether the compound binds to said phage protein or peptides and peptide fragments thereof in said sample. In a preferred embodiment the compound is an antibody.

According to another aspect, a method for detecting the presence of a nucleic acid molecule of the invention is disclosed as comprising the steps of:contacting the sample with a nucleic acid probe or primer which selectively hybridizes to the nucleic acid molecule; and determining whether the nucleic acid probe or primer binds to a nucleic acid molecule in the sample.

According to another aspect of the invention, a kit is disclosed that contains a compound which selectively binds to a phage protein or peptides and peptide fragments thereof of the invention and instructions for use. In a preferred embodiment, a kit is disclosed that contains a compound which selectively hybridizes to a nucleic acid molecule of the invention and and instructions for use.

According to another aspect, the invention discloses a drug screening method for identifying a compound which binds to a polypeptide of the invention comprising the steps of: contacting a polypeptide, or a cell expressing a polypeptide of the invention with a test compound; and determining whether the polypeptide binds to the test compound. The drug screening method also includes methods for modulating the activity of a polypeptide of the invention, as disclosed and described herein, comprising contacting a polypeptide or a cell expressing a polypeptide of the invention with a compound which binds to the polypeptide in a sufficient concentration to modulate the activity of the polypeptide.

### BRIEF DESCRIPTION OF THE DRAWINGS

FIG. 1 is an electron micrograph of group A streptococci treated with lysin showing the collapse of the cell wall and the cell contents pouring out;

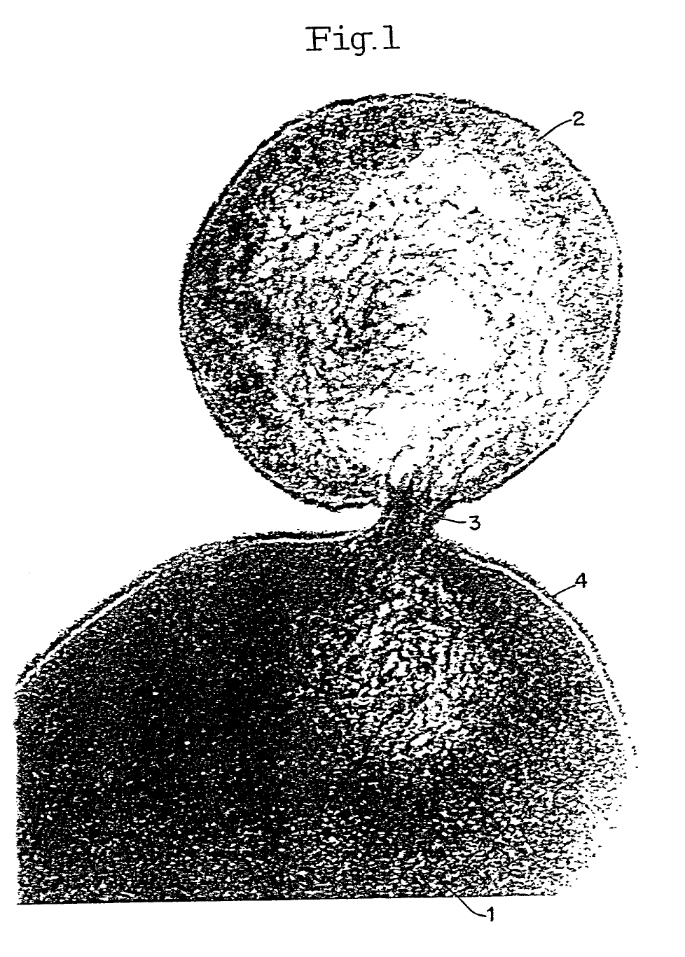

FIG. 2 is a graph for the killing of S. pneumoniae (#DCC 1490) serotype 14 with PAL at various dilutions;

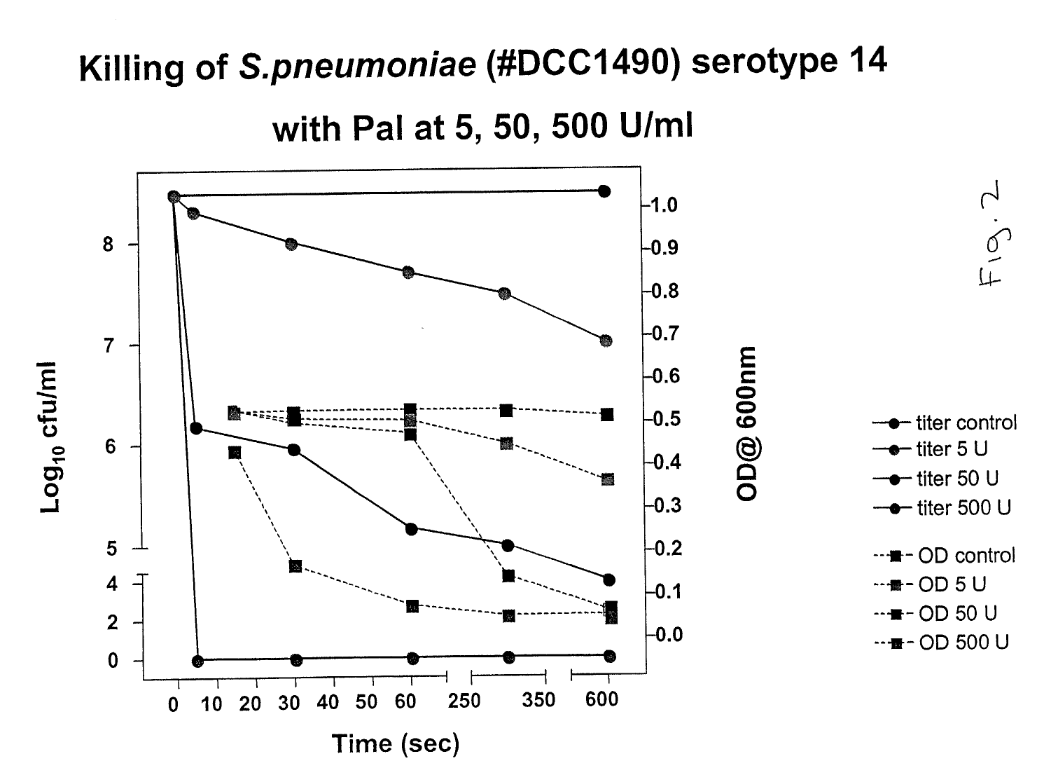

FIG. 3 is a graph showing the decrease of bacterial titer within 30 seconds after addition of 100 U Pal phage enzyme;

FIG. 4 is a series of graphs showing the decrease of the Bacterial titer with 30 seconds after the addition of 100, 1,000, and 10,000 U Pal Lytic Enzyme; and

FIG. 5 is a series of graphs showing the decrease of bacterial titer within 30 seconds after addition of different amounts of U Pal.

FIG. 6 is depicts a histogram showing Group A Streptococci, Group B to N Streptococci, and oral Strptoccocci. The optical density of different strains of bacteria at OD650/min. weremeasured.against different concentration of Pal enzyme.

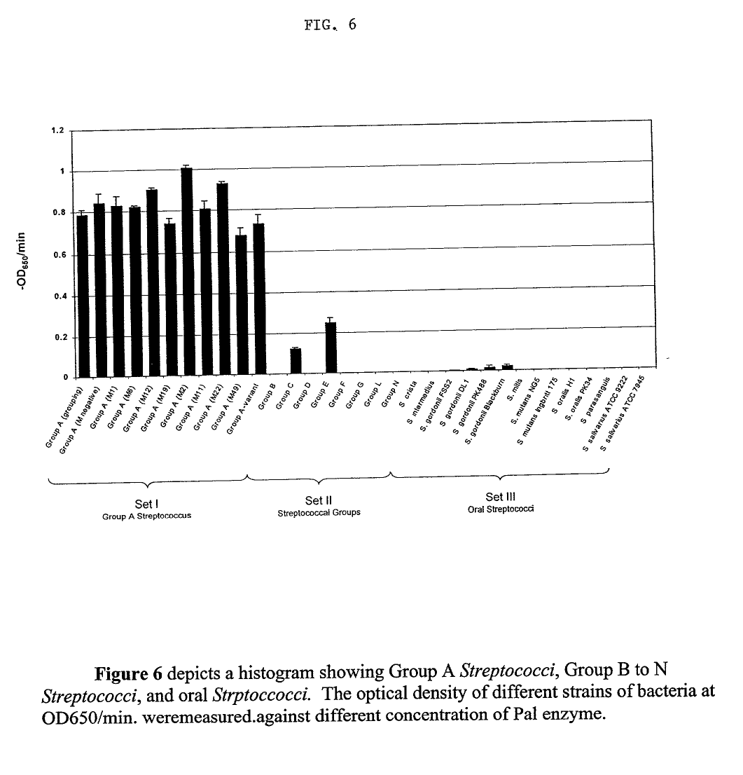

FIG. 7 shows polyacrylamide gel showing molecular weight of a lysin peptide.

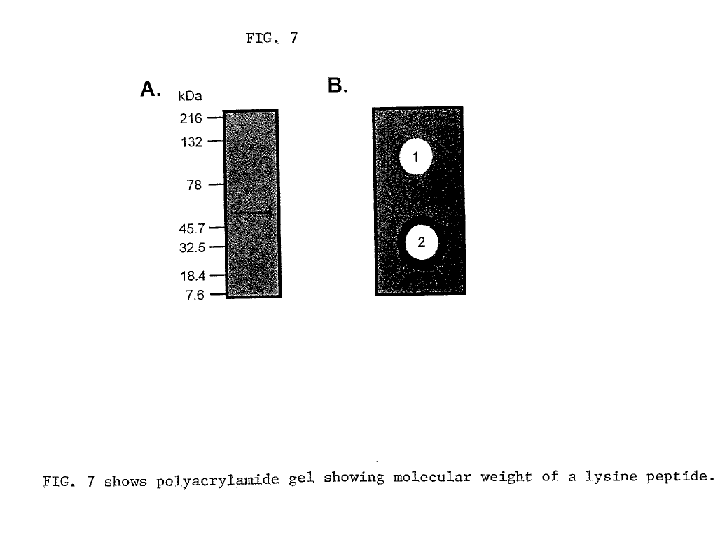

### DETAILED DESCRIPTION OF THE INVENTION

The active drug of the invention, as described herein, includes one or more bacteria-associated phage protein or peptides and peptide fragments thereof. Bacteria-associated phage protein, as disclosed herein, includes variety of bacteria-specific phage lysin and holin proteins that are derived from one or several bacterial species.

Bacteriophage lytic proteins are proteins that specifically cleave bonds that are present in the peptidoglycan of bacterial cells. Since the bacterial cell wall pepidoglycan is highly conserved among all bacterial, there are only a few bonds to be cleaved to disrupt the cell wall. Proteins that cleave these bonds are either muramidases, glucossaminidases, endopeptidases, or N-acetyl-muramoyl-L-alanine amidases (or amidases). The majority of reported phage proteins are either muramidases or amidases, and there have been no reports of bacteriophage glucosaminidases. Fischetti et al (1974) reported that the C1 streptococal phage lysine protein was an amidase. Garcia et al (1987, 1990) reported that the CP-1 lysin from a S. pneumoniae phage was a murmidase. Caldentey and Bamford (1992) reported that a lytic protein from the phi 6 Pseudominas phage was an endopeptidase, splitting the peptide bridge formed by meso-diaminopimilic acid and D-alanine. The E.coli T1 and T6 phage lytic proteins are amidases as is the lytic protein from Listeria phage (ply) (Loessner et al 1996).

Infection of the Hemophilus bacteria by Bacteriophage HP1 (a member of the P2-like phage family with strong similarities to coliphages P2 and 186, and some similarity to the retronphage Ec67) produces a lytic protein capable of lysing the bacteria. The lytic protein for Streptococcus pneumoniae, previously identified as an N-acetyl-muramoyl-L-alanine amidase, is produced by infecting Streptococcus pneumoniae with the Pal bacteriophage. The therapeutic composition contains either or both of the lytic proteins produced by these two bacteria, and also contain other lytic proteins from other bacteria.

Proteins that have the ability to hydrolyze components of a bacterial peptidoglycan fall into one of four categories:

1. N-acetylmuramoyl-L-alanine ainidases (E.C. 3.5.1.28), These proteins hydrolyze the link between N-acetylmuramoyl residues and L-amino acid residues in certain bacterial cell-wall glycopeptides.

Streptococcal lysin belongs to this family of lytic proteins. Of the 27 sequenced amidases, only the 5 highlighted are of bacteriophage origin. The rest are autolysins of bacterial origin.

2. Lysozyme. (EC 3.2.1.17) Also known as Muramidase. This protein hydrolyses the 1,4-beta-linkages between N-acetyl-D-glucosamine and N-acetylmuramic acid in peptidoglycan heteropolymers of the prokaryotes cell walls.

Of the 94 known sequences, 15 are encoded by bacteriophages,.

3. Beta 1,4 N-acetyl-D-glucosaminidase (EC 3.2.1.14) Also known as Chitinase or Chitodextrinase. Hydrolysis of the 1,4-beta-linkages of N-acetyl-D-glucosamine polymers of chitin.

These proteins are found primarily in the plant kingdom, although some are found in bacteria. None of the 104 known proteins are encoded by bacteriophages. However, many of these proteins that are produced by bacteria also possess a lysozyme activity, and are usually classified with the other lysozymcs.

4. Endopeptidase that cleaves the cross bridge of the peptidoglycan. The only known endopeptidase to be characterized extensively which acts on the peptidoglycan is lysostaphin (EC 3.4.24.75) This is a metalloprotease that hydrolyses the -Gly-1-Glybond in the pentaglycine inter-peptide link joining staphylococcal cell wall peptidoglycans. This protein is found several streptococcal species, but it is not encoded by bacteriophages. The only reported phage encoded endopeptidase that acts on the peptidoglycan is from a Pseudomonas phi 6 phage.

Treatment with lytic proteins are shown to be faster and more efficient than with antibiotics. These proteins are specifically effective in prophylatic and therapeutic treatment of bacterial infection of the upper respiratory tract. The infection can be prophylactically or therapeutically treated with a composition comprising an effective amount of at least one lytic protein produced by a bacteria being infected with a bacteriophage specific for that bacteria, and an application means for delivering the lytic protein to the cite of the infection, for example, mouth, throat, or nasal passage.

For example, Streptococcus group A that produces what is commonly known as “strep” throat is treated prophylactically and therapeutically by the application of lytic proteins. When group C Streptococci are infected with a C1 bacteriophage, a lytic protein is produced specific for the lysing of Streptococcus group A. The composition used for the prophylactic and therapeutic treatment of a strep infection includes, for example, one or more lytic proteins and a pharmaceutically acceptable carrier to the mucosal lining of the oral and nasal cavity, such that the protein reaches the mucosa lining.

Another example of a bacteria-associated phage protein used in the composition of this invention is the holin proteins. Holin proteins produce holes in the cell membrane. More specifically, holins form lethal membrane lesions that terminate respiration. Like the lytic proteins, holin proteins are coded for and carried by a phage. In fact, it is quite common for the genetic code of the holin protein to be next to or even within the code for the phage lytic protein. Most holin protein sequences are short, and overall, hydrophobic in nature, with a highly hydrophilic carboxy-terminal domain. In many cases, the putative holin protein is encoded on a different reading frame within the enzymatically active domain of the phage. In other cases, holin protein is encoded on the DNA next or close to the DNA coding for the cell wall lytic protein. Holin proteins are frequently synthesized during the late stage of phage infection and found in the cytoplasmic membrane where they cause membrane lesions

Holins can be grouped into two general classes based on primary structure analysis. Class I holins are usually 95 residues or longer and may have three potential transmembrane domains. Class II holins are usually smaller, at approximately 65-95 residues, with the distribution of charged and hydrophobic residues indicating two TM domains (Young, et al. Trends in Microbiology v. 8, No. 4, March 2000). At least for the phages of gram-positive hosts, however, the dual-component lysis system may not be universal. Although the presence of holins has been shown or suggested for several phages, no genes have yet been found encoding putative holins for all phages. Holins have been shown to be present in several bacteria, including, for example, lactococcal bacteriophage Tuc2009, lactococcal NLC3, pneumococcal bacteriophage EJ-1, Lactobacillus gasseri bacteriophage Nadh, Staphylococcus aureus bacteriophage Twort, Listeria monocytogenes bacteriophages, pneumococcal phage Cp-1, Bacillus subtillis phage M29, Lactobacillus delbrueckki bacteriophage LL-H lysin, and bacteriophage N11 of Staphyloccous aureus. (Loessner, et al., Journal of Bacteriology, Aug. 1999, p. 4452-4460).

There are a large number of phages which will attach to specific bacteria and produce proteins which will lyse that particular bacteria. The following are a list of bacteriophages and bacteria for which they are specific. It is noted that the bacteri and bacteriophages of the invention is not limited to the list disclosed below.

Bacteriophages

Streptococci, Pseudomonas, Pneumococci, Salmonella, Staphylococci, Shigella, Haemophilus, Listeria, Mycobacteria, Vibrio, Corynebacteria, Bacillus, Spirochete, Myxococcus, Burkholderia, Brucella, Yersinia, Clostridium, Campylobacter, Neisseria, Actinomycetes, Agrobacterium, Alcaligenes, Clostridium, Coryneforms, Cyanobacteria, Enterobacteria, Lactobacillus, Lactoctococcus, Micrococcus, Pasteurella, Rhizobium, Xanthomonas, Bdellovibrio, mollicutes, Chiamydia, Spiroplasma, Caulobacter

Various phages which can be used to infect these bacteria and create the lytic protein include:

Actinomycetes, A1-Dat, Bir, M1, MSP8, P-a-1, R1, R2, SV2,VP5, PhiC, φ31C, φUW21, φ15-A, φ150A, 119, SK1, 108/016

Aeromonas, 29, 37,43, 51, 59.1

Altermonas, PM2

Bacillus, AP50, φNS11, BLE, Ipy-1, MP15, mor1, PBP1, SPP1, Spbb, type F, alpha, φ105, 1A, II, Spy-2, SST, G, MP13, PBS1, SP3, SP8, SP10, SP15, SP50

Bdellovibrio, MAC-1, MAC-1′, MAC-2, MAC-4, MAC-4′, MAC-5, MAC-7

Caulobacter, φCb2, φCb4, φCb5, φCb8r, φCb9, φCB12r, φCb23r, φCP2, φCP18, φCr14, φCr28, PP7, φCb2, φCb4, φCb5, φCb8r, φCb9, φCB12r, φCb23r, φCP2, φCP18, φCr14, φCr28, PP7

Chlamydia, Chp-1

Clostridium, F1, HM7, HM3, CEB,

Coliform, AE2, dA, Ec9, f1 , fd, HR, M13, ZG/2, ZJ/2

Coryneforms, Arp, BL3, CONX, MT, Beta, A8010, A19

Cyanobacteria, S-2L, S-4L, N1, AS-1, S-6(L)

Enterobacter, C-2, If1, If2, Ike, I2-2, PR64FS, SF, tf-1, PRD1, H-19J, B6, B7, C-1, C2, Jersey, ZG/3A, T5, ViII, b4, chi, Beccles, tu, PRR1, 7s, C-1, c2, fcan, folac, Ialpha, M, pilhalpha, R23, R34, ZG/1, ZIK/1, ZJ/1, ZL/3, ZS/3, alpha15, f2, fr, FC3-9, K19, Mu, 01, P2, ViI, φ92, 121, 16-19, 9266, C16, DdVI, PST, SMB, SMP2, a, 3, 3T+, 9/0, 11F, 50, 66F, 5845, 8893, M11, QB, ST, TW18, VK, FI, ID2, fr, f2,

Listeria, H387, 2389, 2671, 2685, 4211

Micrococcus, N1, N5

Mycobacterium, Lacticola, Leo, R1-Myb, 13

Pasteurella, C-2, 32, AU

Pseudomonas, Phi6, Pf1 , Pf2, Pf3, D3, Kf1, M6, PS4, SD1, PB-1, PP8, PS17, nKZ, nW-14, n1, 12S,

Staphyloccous, 3A, B11-M15, 77, 107, 187, 2848A, Twort

Streptococcus, A25, A25 PE1, A25 VD13, A25 omega8, A25 24

Steptococcus A

Vibrio, OXN-52P, VP-3, VP5, VP11, alpha3alpha, IV, kappa, 06N-22-P, VP1, x29, II, nt-1,

Xanthomonas, Cf; Cf1t, Xf, Xf2, XP5

The composition of this invention contains phage peptides and peptide fragments thereof as well as, or instead of phage proteins.

Phage Protein, as disclosed herein, includes phage polypeptides, peptide fragments, nucleic acid molecules encoding phage protein or protein peptides fragments, antibody and antibody fragments, having biological activity either alone or with combination of other molecules

Nucleic acid molecules, as disclosed herein includes genes, gene fragments polynucleotides, oligonucleotides, DNA, RNA, DNA-RNA hybrids, EST, SNIPs, genomic DNA, cDNA, mRNA,, antisense RNA, Ribozymes vectors containing nucleic acid molecules, regulatory sequences, signal sequences. Nucleic acid molecules of this invention include any nucleic acid-based molecule that either alone or in combination with other molecules produce an oligonucleotide molecule capable or incapable of translation into a peptide

The natural form of the protein or peptides fragments, as disclosed herein, includes an “isolated” or “purified” phage protein or peptides fragments, or biologically active portion thereof that is substantially free of cellular material or other contaminating proteins from the cell or tissue source from which the protein is derived, or substantially free of chemical precursors or other chemicals when isolated. The language “substantially free of cellular material” includes preparations of protein in which the protein is separated from cellular components of the host bacteria from which it is isolated. Thus, protein or peptides and peptide fragments thereof that is substantially free of bacterial material includes preparations of protein or peptides and peptide fragments thereof having less than about 30%, 20%, 10%, or 5% (by dry weight) of heterologous protein (also referred to herein as a “contaminating protein”).

The modified from of the protein or peptides and peptide fragments, as disclosed herein, includes, protein or peptides and peptide fragments that are chemically synthesized or prepared by recombinant DNA techniques, or both. These techniques include, for example, chimerization and shuffling. When the protein or peptide is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

The invention also provides chimeric proteins or peptides fragments, which include fusion proteins. Chimeric proteins or peptides are produced, for example, by combining two or more proteins having two or more active sites. Chimeric protein and peptides can act independently on the same or different molecules, and hence have a potential to treat two or more different bacterial infections at the same time. Chimeric proteins and peptides also are used to treat a bacterial infection by cleaving the cell wall in more than one location.

As used herein, a “chimeric protein” or “fusion protein” comprises all or (preferably a biologically active) part of a polypeptide of the invention operably linked to a heterologous polypeptide. The term “operably linked” means that the polypeptide of the invention and the heterologous polypeptide are fused in-frame. The heterologous polypeptide can be fused to the N-terminus or C-terminus of the polypeptide of the invention. Chimeric proteins are produced enzymatically by chemical synthesis, or by recominant DNA technology.

One useful fusion protein is a GST fusion protein in which the polypeptide of the invention is fused to the C-terminus of a GST sequence. Such chimeric protein can facilitate the purification of a recombinant polypeptide of the invention.

In another embodiment, the chimeric protein or peptide contains a heterologous signal sequence at its N-terminus. For example, the native signal sequence of a polypeptide of the invention can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a polypeptide of the invention is fused to sequences derived from a member of the immunoglobulin protein family. An immunoglobulin fusion protein of the invention can be incorporated into a pharmaceutical composition and administered to a subject to inhibit an interaction between a ligand (soluble or membrane-bound) and a protein on the surface of a cell (receptor), to thereby suppress signal transduction in vivo. The immunoglobulin fusion protein can alter bioavailability of a cognate ligand of a polypeptide of the invention. Inhibition of ligand/receptor interaction may be useful therapeutically, both for treating bacterial-associated diseases and disorders for modulating (i.e. promoting or inhibiting) cell survival. Moreover, an immunoglobulin fusion protein of the invention can be used as an immunogen to produce antibodies directed against a polypeptide of the invention in a subject, to purify ligands and in screening assays to identify molecules which inhibit the interaction of receptors with ligands.

Chimeric and fusion proteins and peptides of the invention can be produced by standard recombinant DNA techniques. In another embodiment, the fusion gene can be synthesized by conventional techniques, including automated DNA synthesizers. Alternatively, PCR amplification of gene fragments can be carried out using anchor primers which give rise to complementary overhangs between two consecutive gene fragments which subsequently can be annealed and reamplified to generate a chimeric gene sequence (see, i.e., Ausubel et al., supra). Moreover, many expression vectors are commercially available that already encode a fusion moiety (i.e., a GST polypeptide). A nucleic acid encoding a polypeptide of the invention can be cloned into such an expression vector such that the fusion moiety is linked in-frame to the polypeptide of the invention.

As used herein, shuffled proteins or peptides are molecules in which the genes, gene products, or peptides for more than one related phage protein or protein peptide fragments have been randomly cleaved and reassembled into a more active or specific protein. Shuffled oligonucleotides, peptides or peptide fragment molecules are selected or screened to identify a molecule having a desired functional property. This method is described, for example, in Stemmer, U.S. Pat. No. 6,132,970.(Method of shuffling polynucleotides); Kauffinan, U.S. Pat. No 5, 976,862 (Evolution via Condon-based Synthesis) and Huse, U.S. Pat. No. 5,808,022 (Direct Codon Synthesis). The contents of these patents are incorporated herein by reference.

Shuffling is used to create a protein that is 10 to 100 fold more active than the template protein. The template protein is selected among different varieties of lysin or holin proteins. The shuffled protein or peptides constitute, for example, one or more binding domains and one or more catalytic domains. Each binding or catalytic domain is derived from the same or a different phage or phage protein. The shuffled domains are either oligonucleotide based molecules, as gene or gene products, that either alone or in combination with other genes or gene products are translatable into a peptide fragment, or they are peptide based molecules. Gene fragments include any molecules of DNA, RNA, DNA-RNA hybrid, antisense RNA, Ribozymes, ESTs, SNIPs and other oligonucleotide-based molecules that either alone or in combination with other molecules produce an oligonucleotide molecule capable or incapable of translation into a peptide.

In addition, libraries of fragments of the coding sequence of a polypeptide of the invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan (1992) Proc. Natl. Acad. Sci. USA 89:7811-7815; Delgrave et al. (1993) Protein Engineering 6(3):327-331).

Biologically active portions of a protein or peptide fragment of the invention, as described herein, include polypeptides comprising amino acid sequences sufficiently identical to or derived from the amino acid sequence of the phage protein of the invention, which include fewer amino acids than the full length protein of the phage protein and exhibit at least one activity of the corresponding full-length protein. Typically, biologically active portions comprise a domain or motif with at least one activity of the corresponding protein. A biologically active portion of a protein or protein fragment of the invention can be a polypeptide which is, for example, 10, 25, 50, 100 less or more amino acids in length. Moreover, other biologically active portions, in which other regions of the protein are deleted, or added can be prepared by recombinant techniques and evaluated for one or more of the functional activities of the native form of a polypeptide of the invention.

A signal sequence of a polypeptide of the invention can facilitate transmembrane movement of the protein and peptides and peptide fragments of the invention to and from mucous membranes, as well as by facilitating secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to the signal sequence itself and to the polypeptide in the absence of the signal sequence (i.e., the cleavage products).

In one embodiment, a nucleic acid sequence encoding a signal sequence of the invention can be operably linked in an expression vector to a protein of interest, such as a protein which is ordinarily not secreted or is otherwise difficult to isolate. The signal sequence directs secretion of the protein, such as from an eukaryotic host into which the expression vector is transformed, and the signal sequence is subsequently or concurrently cleaved. The protein can then be readily purified from the extracellular medium by art recognized methods. Alternatively, the signal sequence can be linked to a protein of interest using a sequence which facilitates purification, such as with a GST domain.

In another embodiment, a signal sequence of the present invention can be used to identify regulatory sequences, i.e., promoters, enhancers, repressors. Since signal sequences are the most amino-terminal sequences of a peptide, it is expected that the nucleic acids which flank the signal sequence on its amino-terminal side will be regulatory sequences that affect transcription. Thus, a nucleotide sequence which encodes all or a portion of a signal sequence can be used as a probe to identify and isolate the signal sequence and its flanking region, and this flanking region can be studied to identify regulatory elements therein.

The present invention also pertains to variants of the polypeptides of the invention. Such variants have an altered amino acid sequence which can function as either agonists (mimetics) or as antagonists. Variants can be generated by mutagenesis, i.e., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein of the invention which function as either agonists (mimetics) or as antagonists can be identified by screening combinatorial libraries of mutants, i.e., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (i.e., for phage display). There are a variety of methods which can be used to produce libraries of potential variants of the polypeptides of the invention from a degenerate oligonucleotide sequence. Methods for synthesizing degenerate oligonucleotides are known in the art (see, i.e., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

A phage protein or peptide fragment of this invention can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (preferably 10, 15, 20, or 30) amino acid residues of the amino acid sequence of a phage protein or protein peptide fragments of the invention. and encompasses an epitope of the protein such that an antibody raised against the peptide forms a specific immune complex with the protein.

An immunogen typically is used to prepare antibodies by immunizing a suitable subject, (i.e., rabbit, goat, mouse or other mammal). An appropriate immunogenic preparation can contain, for example, recombinantly expressed or chemically synthesized polypeptide. The preparation can further include an adjuvant, such as Freund's complete or incomplete adjuvant, or similar immunostimulatory agent.

Accordingly, another aspect of the invention pertains to antibodies directed against a polypeptide of the invention. The term “antibody” as used herein refers to immunoglobulin molecules and immunologically active portions of immunoglobulin molecules, i.e., molecules that contain an antigen binding site which specifically binds an antigen, such as a polypeptide of the invention, i.e., an epitope of a polypeptide of the invention. A molecule which specifically binds to a given polypeptide of the invention is a molecule which binds the polypeptide, but does not substantially bind other molecules in a sample, i.e., a biological sample, which naturally contains the polypeptide. Examples of immunologically active portions of immunoglobulin molecules include F(ab) and F(ab′)₂ fragments which can be generated by treating the antibody with an protein such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. Preferred polyclonal antibody compositions are ones that have been selected for antibodies directed against a phage protein or protein peptide fragments of the invention. Particularly preferred polyclonal antibody preparations are ones that contain only antibodies directed against a polypeptide or polypeptides of the invention. Particularly preferred immunogen compositions are those that contain no other human proteins such as, for example, immunogen compositions made using a non-human host cell for recombinant expression of a polypeptide of the invention. In such a manner, the only human epitope or epitopes recognized by the resulting antibody compositions raised against this immunogen will be present as part of a polypeptide or polypeptides of the invention

All isozymes, variants or analogs of the bacteri-associated phage proteins and peptides and peptide fragments of the invention, whether natural or modified, are encompassed and included within the scope of the invention.

Methods of Treatment

The present invention provides for both prophylactic and therapeutic methods of treating a subject at risk of (or susceptible to) a disorder or having a disorder associated with aberrant or excessive r activity of a phage protein or protein peptide fragments of the invention. For example, disorders characterized by abberant expression or activity of the phage protein or peptides and peptide fragments of the invention include bacterial associated disease and disorders.

In particular, the invention features the use of the bacteria-associated lytic and holin proteins, or peptides and peptide fragments thereof in the therapeutic composition and method disclosed. These proteins used, are derived from a variety of bacterial species and subspecies. More comprehensive list of these bacteria and their associated proteins are disclosed above. The examples of bacteria that causes infectious disease includes, Streptococcal pygenes, Hemophilus influenza, Pseudomonas, Streptococcus pneumoniae, Streptococcus fasciae, Streptococcus group B, Listeria, Salmonella, E. coli, Campylobacter, Mycobacteria tuberculosis Staphylococcu, or a combination thereof.

Prophylactic Methods

In one aspect, the invention provides a method for preventing in a subject, a disease or condition associated with an aberrant expression or activity of an protein or protein peptide fragments of the invention, by administering to the subject an agent which modulates expression or at least one activity of the protein or protein peptide fragments. Subjects at risk for a disease which is caused or contributed to by aberrant expression or activity of a polypeptide of the invention can be identified by, for example, any or a combination of diagnostic or prognostic assays as described herein. Administration of a prophylactic agent can occur prior to the manifestation of symptoms characteristic of the aberrancy, such that a disease or disorder is prevented or, alternatively, delayed in its progression. Depending on the type of aberrancy, for example, an agonist or antagonist agent can be used for treating the subject. The appropriate agent can be determined based on screening assays described herein.

Methods of Detection

The invention also encompasses kits for detecting the presence of a polypeptide or nucleic acid of the invention in a biological sample (a test sample). Such kits can be used to determine if a subject is suffering from or is at increased risk of developing a disorder associated with aberrant expression of a polypeptide of the invention (i.e., a bacterial-related disease or disorder). For example, the kit can comprise a labeled compound or agent capable of detecting the polypeptide or mRNA encoding the polypeptide in a biological sample and means for determining the amount of the polypeptide or mRNA in the sample (i.e., an antibody which binds the polypeptide or an oligonucleotide probe which binds to DNA or mRNA encoding the polypeptide). Kits may also include instructions for observing that the tested subject is suffering from or is at risk of developing a disorder associated with aberrant expression of the polypeptide if the amount of the polypeptide or MRNA encoding the polypeptide is above or below a normal level.

For antibody-based kits, the kit may comprise, for example: (1) a first antibody (i.e., attached to a solid support) which binds to a polypeptide of the invention; and, optionally, (2) a second, different antibody which binds to either the polypeptide or the first antibody and is conjugated to a detectable agent.

For oligonucleotide-based kits, the kit may comprise, for example: (1) an oligonucleotide, i.e., a detectably labeled oligonucleotide, which hybridizes to a nucleic acid sequence encoding a polypeptide of the invention or (2) a pair of primers useful for amplifying a nucleic acid molecule encoding a polypeptide of the invention. The kit may also comprise, i.e., a buffering agent, a preservative, or a protein stabilizing agent. The kit may also comprise components necessary for detecting the detectable agent (i.e., an protein or a substrate). The kit may also contain a control sample or a series of control samples which can be assayed and compared to the test sample contained. Each component of the kit is usually enclosed within an individual container and all of the various containers are within a single package along with instructions, typically a manual, for observing whether the tested subject is suffering from or is at risk of developing a disorder associated with aberrant expression of the polypeptide.

Method of Drug Screening

The invention, described herein, features drug screening methods for identifying a compound which binds to a polypeptide of the invention. The drug screening method includes contacting a polypeptide, or a cell expressing a polypeptide of the invention with a test compound; and determining whether the polypeptide binds to the test compound. The drug screening method also includes methods for modulating the activity of a polypeptide of the invention, as disclosed and described herein, comprising contacting a polypeptide or a cell expressing a polypeptide of the invention with a compound which binds to the polypeptide in a sufficient concentration to modulate the activity of the polypeptide.

Complementary agents

In order to accelerate treatment of the infection, the therapeutic composition may further include at least one complementary agent, which can also potentiate the bactericidal activity of the phage lytic or holin proteins. These complementary agents include, for example, antimicrobial agents, anti-inflammatory agents, antiviral agents, local anesthetic agents, corticosteroids, destructive therapy agents, antifungals, antiandrogens, or a combination thereof. Specific examples of the complementary agent include, dapsone, erythromycin, minocycline, tetracycline, clindamycin, penicillin, synthetic penicillins bacitracin, methicillin, cephalosporin, polymyxin, cefaclor. Cefadroxil, cefamandole nafate, cefazolin, cefixime, cefinetazole, cefonioid, cefoperazone, ceforanide, cefotanme, cefotaxime, cefotetan, cefoxitin, cefpodoxime proxetil, ceftazidime, ceftizoxime, ceftriaxone, cefriaxone moxalactam , cefuroxime, cephalexin, cephalosporin C, cephalosporin C sodium salt, cephalothin, cephalothin sodium salt, cephapirin, cephradine, cefuroximeaxetil, dihydratecephalothin, moxalactam, loracarbef. Mafate.

The preferred concentration for antimicrobials is from about 0.5% to about 10% by weight of the total composition.

Local anesthetics include tetracaine, tetracaine hydrochloride, lidocaine, lidocaine hydrochloride, dyclonine, dyclonine hydrochloride, dimethisoquin hydrochloride, dibucaine, dibucaine hydrochloride, butambenpicrate, and pramoxine hydrochloride. A preferred concentration for local anesthetics is from about 0.025% to 5% by weight of the total composition. Anesthetics such as benzocaine is also be used at a preferred concentration of from about 2% to 25% by weight. Corticosteroids includes betamethasone dipropionate, fluocinolone actinide, betamethasone valerate, triamcinolone actinide, clobetasol propionate, desoximetasone, diflorasone diacetate, amcinonide, flurandrenolide, hydrocortisone valerate, hydrocortisone butyrate, and desonide are recommended at concentrations of from about 0.01% to 1.0% by weight. Preferred concentrations for corticosteroids such as hydrocortisone or methylprednisolone acetate are from about 0.2% to about 5.0% by weight.

Destructive therapy agents such as salicylic acid or lactic acid are also used as complementary agents. A concentration of from about 2% to about 40% by weight of the total concentration is preferred. Cantharidin is preferably utilized in a concentration of from about 5% to about 30% by weight. Typical antifungals that may be used in this invention and their preferred weight concentrations include: oxiconazole nitrate (0.1% to 5.0%), ciclopirox olamine (0.1% to 5.0%), ketoconazole (0.1% to 5.0%), miconazole nitrate (0.1% to 5.0%), and butoconazole nitrate (0.1% to 5.0%).

For the topical treatment of seborrheic dermatitis, hirsutism, acne, and alopecia, the active agent includes an antiandrogen such as flutamide or finasteride in preferred weight percentages of about 0.5% to 10%.

Typically, treatments using a combination of drugs include antibiotics in combination with local anesthetics such as polymycin B sulfate and neomycin sulfate in combination with tetracaine for topical antibiotic gels to provide prophylaxis against infection and relief of pain. Another example is the use of minoxidil in combination with a corticosteroid such as betamethasone diproprionate for the treatment of alopecia ereata. The combination of an anti-inflammatory such as cortisone with an antifungal such as ketoconazole for the treatment of tinea infections is also an example.

Additionally, the complementary agent may further comprise the protein lysostaphin for the treatment of any Staphylococcus aureus bacteria. Mucolytic peptides, such as lysostaphin, have been suggested to be efficacious in the treatment of S. aureus infections of humans (Schaffner et al., Yale J. Biol. & Med., 39:230 (1967) and bovine mastitis caused by S. aureus (Sears et al., J. Dairy Science, 71 (Suppl. 1): 244(1988)). Lysostaphin, a gene product of Staphylococcus simulans, exerts a bacteriostatic and bactericidal effect upon S. aureus by enzymatically degrading the polyglycine crosslinks of the cell wall (Browder et al., Res. Comm., 19: 393-400 (1965)). U.S. Pat. No. 3,278,378 describes fermentation methods for producing lysostaphin from culture media of S. staphylolyticus, later renamed S. simulans. Other methods for producing lysostaphin are further described in U.S. Pat. Nos. 3,398,056 and 3,594,284. The gene for lysostaphin has subsequently been cloned and sequenced (Recsei et al., Proc. Natl. Acad. Sci. USA, 84: 1127-1131 (1987)). The recombinant mucolytic bactericidal protein, such as r-lysostaphin, can potentially circumvent problems associated with current antibiotic therapy because of its targeted specificity, low toxicity and possible reduction of biologically active residues.

Furthermore, lysostaphin is also active against non-dividing cells, while most antibiotics require actively dividing cells to mediate their effects (Dixon et al., Yale J. Biology and Medicine, 41: 62-68 (1968)). Lysostaphin, in combination with the phage proteins, can be used in the presence or absence of the listed antibiotics. There is a degree of added importance in using both lysostaphin and the phage proteins in the same therapeutic composition. Frequently, when a body has a bacterial infection, the infection by one genus of bacteria weakens the body or changes the bacterial flora of the body, allowing other potentially pathogenic bacteria to infect the body. One of the bacteria that sometimes co-infects the body is Staphylococcus aureus. Many strains of Staphylococcus aureus produce penicillinase, such that Staphylococcus, Streptococcus, and other gram positive bacterial strains will not be killed by standard antibiotics. Consequently, the use of the lysin and lysostaphin, possibly in combination with antibiotics, can serve as the most rapid and effective treatment of bacterial infections. Other examples of a complementary agent include mutanolysin, and lysozyme

To enforce and increase site or tissue specificity of the phage proteins, appropriate site-specific promoters or other molecules, polynucleotide, peptide, or non-peptide based, may be attached to the phage protein in a proper orientation. These molecules ideally ease the transport of proteins across the cell membrane to the site of the bacteria.

According to an embodiment of the invention, the phage proteins, or their peptide fragments are directed to the mucosal lining, where, in residence, they kill colonizing disease bacteria.

Mucosal lining, as disclosed and described herein, includes, for example, the cul-de-sac of the eye, buccal cavity, nose, rectum, vagina, periodontal pocket, intestines and colon. Due to natural eliminating or cleansing mechanisms of mucosal tissues, conventional dosage forms are not retained at the application site for any significant length of time. For example, drops of medications instilled in the cul-de-sac of the eye are easily eliminated; first, by overflowing, and subsequently, by drainage through puncta. Conventional vaginal dosage forms such as creams, ointments, suppositories, etc, are rapidly removed by self cleansing action of the vaginal tract.

For these and other reasons it is advantageous to have materials which exhibit adhesion to mucosal tissues, to be administered with one or more phage protein and other complementary agents over a period of time. Materials having controlled release capability are particularly desirable, and the use of sustained release mucoadhesives has received a significant degree of attention.

J. R. Robinson (U.S. Pat. No. 4,615,697) provides a good review of the various controlled release polymeric compositions used in mucosal drug delivery. The patent describes a controlled release treatment composition which includes a bioadhesive and an effective amount of a treating agent. The bioadhesive is a water swellable, but water insoluble fibrous, crosslinked, carboxy functional polymer containing (a) a plurality of repeating units of which at least about 80 percent contain at least one carboxyl functionality, and (b) about 0.05 to about 1.5 percent crosslinking agent substantially free from polyalkenyl polyether. While the polymers of Robinson are water swellable but insoluble, they are crosslinked, not thermoplastic, and are not as easy to formulate with active agents, and into the various dosage forms, as the copolymer systems of the present application.

Other approaches involving mucoadhesives which are the combination of hydrophilic and hydrophobic materials, are known. Orahesive.RTM. from E.R. Squibb & Co is an adhesive which is a combination of pectin, gelatin, and sodium carboxymethyl cellulose in a tacky hydrocarbon polymer, for adhering to the oral mucosa. However, such physical mixtures of hydrophilic and hydrophobic components eventually fall apart. In contrast, the hydrophilic and hydrophobic domains in the present invention produce an insoluble copolymer.

U.S. Pat. No. 4,948,580 describes a bioadhesive oral drug delivery system. The composition, includes a freeze-dried polymer mixture formed of the copolymer poly(methyl vinyl ether/maleic anhydride) and gelatin, dispersed in an ointment base, such as mineral oil containing dispersed polyethylene. U.S. Pat. No. 5,413,792 discloses paste-like preparations comprising (A) a paste-like base comprising a polyorganosiloxane and a water soluble polymeric material which are preferably present in a ratio by weight from 3:6 to 6:3, and (B) an active ingredient. U.S. Pat. No. 5,554,380 discloses a solid or semisolid bioadherent orally ingestible drug delivery system containing a water-in-oil system having at least two phases. One phase comprises from about 25% to about 75% by volume of an internal hydrophilic phase and the other phase comprises from about 23% to about 75% by volume of an external hydrophobic phase, wherein the external hydrophobic phase is comprised of three components: (a) an emulsifier, (b) a glyceride ester, and (c)a wax material.

U.S. Pat. No. 5,942,243 describes a release material useful for administering antibacterial agents of the invention.

An embodiment of the present invention features therapeutic compositons containing polymeric mucoadhesives consisting essentially of a graft copolymer comprising a hydrophilic main chain and hydrophobic graft chains for controlled release of biologically active agents. The graft copolymer is a reaction product of (1) a polystyrene macromonomer having an ethylenically unsaturated functional group, and (2) at least one hydrophilic acidic monomer having an ethylenically unsaturated functional group. The graft chains consist essentially of polystyrene, and the main polymer chain of hydrophilic monomeric moieties, some of which have acidic functionality. The weight percent of the polystyrene macromonomer in the graft copolymer is between about 1 and about 20% and the weight percent of the total hydrophilic monomer in the graft copolymer is between 80 and 99%, and wherein at least 10% of said total hydrophilic monomer is acidic, said graft copolymer when fully hydrated having an equilibrium water content of at least 90%.

Compositions containing the copolymers gradually hydrate by sorption of tissue fluids at the application site to yield a very soft jelly like mass exhibiting adhesion to the mucosal surface. During the period of time the composition is adhered to the mucosal surface it provides sustained release of the pharmacologically active agent, which is absorbed by the mucosal tissue.

Mucoadhesivity of the compositions of this invention is, to a large extent, produced by the hydrophilic acidic monomers of the chain in the polystyrene graft copolymer. The acidic monomers include, but are not limited to, acrylic and methacrylic acids, 2-acrylamido-2-methyl-propane sulfonic acid, 2-sulfoethyl methacrylate, and vinyl phosphonic acid. Other copolymerizable monomers include, but are not limited to N,N-dimethylacrylamide, glyceryl methacrylate, polyethylene glycol monomethacrylate, etc.

The compositions of the present invention may optionally contain other polymeric materials, such as poly(acrylic acid), poly,-(vinyl pyrrolidone), and sodium carboxymethyl cellulose plasticizers, and other pharmaceutically acceptable excipients in amounts that do not cause deleterious effect upon mucoadhesivity of the composition. The dosage forms of the compositions of this invention can be prepared by conventional methods.

Pharmaceutical Compositions

The phage proteins, peptides and peptide fragments thereof including polyuncleotide molecules, gene or gene products of the phage protein, and antibodies of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Pharmaceutically accepted carriers of the compositions of the present invention comprise for example, semi-solid and gel-like vehicles that include a polymer thickener, water, preservatives, active surfactants or emulsifiers, antioxidants, sun screens, and a solvent or mixed solvent system. U.S. Pat. No. 5,863,560 (Osborne) discusses a number of different carrier combinations which can aid in the exposure of the skin to a medicament. Polymer thickeners that may be used include those known to one skilled in the art, such as hydrophilic and hydroalcoholic gelling agents frequently used in the cosmetic and pharmaceutical industries. Preferably, the hydrophilic or hydroalcoholic gelling agent comprises “CARBOPOL.RTM.” (B. F. Goodrich, Cleveland, Ohio), “HYPAN.RTM.” (Kingston Technologies, Dayton, N.J.), “NATROSOL.RTM.” (Aqualon, Wilmington, Del.), “KLUCEL.RTM.” (Aqualon, Wilmington, Del.), or “STABILEZE.RTM.” (ISP Technologies, Wayne, N.J.). Preferably, the concentration of the gelling agent is from about 0.2% to about 4% by weight of the composition. More preferably, the preferred compositional weight percent range for “CARBOPOL.RTM.” is from about 0.5% to about 2%, while the preferred weight percent range for “NATROSOL.RTM.” and “KLUCEL.RTM.” is from about 0.5% to about 4%. The preferred compositional weight percent range for both “HYPAN.RTM.” and “STABILEZE.RTM.” is from about 0.5% to about 4%.

CARBOPOL.RTM is one of numerous cross-linked acrylic acid polymers that are given the general adopted name carbomer. These polymers dissolve in water and form a clear or slightly hazy gel upon neutralization with a caustic material such as sodium hydroxide, potassium hydroxide, triethanolamine, or other amine bases. “KLUCEL.RTM.” is a cellulose polymer that is dispersed in water and forms a uniform gel upon complete hydration. Other preferred gelling polymers include hydroxyethylcellulose, cellulose gum, MVE/MA decadiene crosspolymer, PVM/MA copolymer, or a combination thereof.

Preservatives, used in this invention as an inactive agent, are preferably in an amount from about 0.05% to 0.5% by weight of the total composition. The use of preservatives assures that the product is not microbially contaminated. The preservatives used in this invention include, methylparaben, propylparaben, butylparaben, chloroxylenol, sodium benzoate, DMDM Hydantoin, 3-Iodo-2-Propylbutyl carbamate, potassium sorbate, chlorhexidine digluconate, or a combination thereof. Titanium dioxide may be used as a sunscreen to serve as prophylaxis against photosensitization. Alternative sunscreens include methyl cinnamate. Moreover, BHA may be used as an antioxidant, as well as to protect ethoxydiglycol and/or dapsone from discoloration due to oxidation. An alternate antioxidant is BHT.

According to a feature of the invention, a mild surfactant is used to potentiate the therapeutic effect of the lytic protein. Suitable mild surfactants include, inter alia, esters of polyoxyethylene sorbitan and fatty acids (Tween series), octylphenoxy polyethoxy ethanol (Triton-X series), n-Octyl-.beta.-D-glucopyranoside, n-Octyl-.beta.-D-thioglucopyranoside, n-Decyl-.beta.-D-glucopyranoside, n-Dodecyl-.beta.-D-glucopyranoside, and biologically occurring surfactants, i.e., fatty acids, glycerides, monoglycerides, deoxycholate and esters of deoxycholate.

In one embodiment, the invention comprises a dermatological composition having from about 0.5% to 10% carbomer and about 0.5% to 10% of a pharmaceutical that exists in both a dissolved state and a micro particulate state. The dissolved pharmaceutical has the capacity to cross the stratum corneum, whereas the micro particulate pharmaceutical does not. Addition of an amine base, potassium, hydroxide solution, or sodium hydroxide solution completes the formation of the gel. More particularly, the composition may include dapsone, an antimicrobial agent having anti-inflammatory properties. A preferred ratio of micro particulate to dissolved dapsone is five or less.

In another embodiment, the composition further includes from about 1% carbomer, about 80-90% water, about 10% ethoxydiglycol, about 0.2% methylparaben, about 0.3% to 3.0% dapsone including both micro particulate dapsone and dissolved dapsone, and about 2% caustic material. More particularly, the carbomer may include “CARBOPOL.RTM. 980” and the caustic material may include sodium hydroxide solution. In a preferred embodiment, the composition comprises dapsone and ethoxydiglycol, which allows for an optimized ratio of micro particulate drug to dissolved drug. This ratio determines the amount of drug delivered, compared to the amount of drug retained in or above the stratum corneum to function in the supracomeum domain. The system of dapsone and ethoxydiglycol may include purified water combined with “CARBOPOL.RTM.” gelling polymer, methylparaben, propylparaben, titanium dioxide, BHA, and a caustic material to neutralize the “CARBOPOL.RTM.”

The invention includes methods for preparing pharmaceutical compositions for modulating the expression or activity of a polypeptide or nucleic acid of the invention. Such methods comprise formulating a pharmaceutically acceptable carrier with an agent which modulates expression or activity of a polypeptide or nucleic acid of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid of the invention and one or more addtional active compounds.

A pharmaceutical composition of the invention is formulated to be compatible with its intended route of administration and means of application.

Examples of routes of administration include parenteral, i.e., intravenous, intradermal, subcutaneous, oral (i e., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (i.e., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, i.e., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (i.e., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Means of application of the pharmaceutical composition, which is also refered to as “carriers” is determined according to the type of the infection and the site of the infection. The means of application include, but are not limited to, suppository enemas, liquid means (for example, syrups, mouthwash, gargles, and eye drops in aqueous or non-aqueous form), solid means (for example, food stuff, confectionary, or toothpaste), drops, ointments, washes, injections, packings, bronchial sprays, aerosols, inhalers, bandages, tampons, and topical creams, among others.

The lozenge, tablet, or gum may contain sugar, corn syrup, a variety of dyes, non-sugar sweeteners, flavorings, any binders, or combinations thereof. Similarly, any gum-based products may contain acacia, carnauba wax, citric acid, corn starch, food colorings, flavorings, non-sugar sweeteners, gelatin, glucose, glycerin, gum base, shellac, sodium saccharin, sugar, water, white wax, cellulose, other binders, and combinations thereof.

Lozenges may further contain sucrose, corn starch, acacia, gum tragacanth, anethole, linseed, oleoresin, mineral oil, and cellulose, other binders, and combinations thereof. Sugar substitutes are also used in place of dextrose, sucrose, or other sugars.

Nasal sprays can be made to have long acting or timed release capabilities, and can be manufactured by means well known in the art. An inhalant may also be used, so that the phage protein may reach further down into the bronchial tract, and lungs.

If alcohol is used in the means of application or carrier, the protein should be in a micelle, liposome, or a “reverse” liposome, to prevent denaturing of the protein. For example, to avoid denaturation of the proteins, mouthwash or similar type products should not contain alcohol. Similarly, when the protein is placed in a cough drop, gum, candy or lozenge during the manufacturing process, such placement should be made prior to the hardening of the lozenge or candy but after the cough drop or candy has cooled somewhat, to avoid heat denaturation of the protein. The protein may be added to these substances in a liquid form or in a lyophilized state, whereupon it will be solubilized when it meets a liquid body

The effective dosage rates or amounts of the page protein to treat the infection, and the duration of treatment depends in part on the seriousness of the infection, the duration of exposure of the recipient to the infectious bacteria, the number of square centimeters of skin or tissue which are infected, the depth of the infection, the objectives of the therapy, and the size and weight of the individual, among others. The duration for use of the composition containing the protein also depends on whether the use is for prophylactic purposes, wherein the use may be hourly, daily or weekly, for a short time period, or whether the use will be for therapeutic purposes wherein a more intensive regimen of the use of the composition may be needed, such that usage may last for hours, days or weeks, and/or on a daily basis, or at timed intervals during the day. Any dosage form employed should provide for a minimum number of units for a minimum amount of time.

The effective concentration of the active units of protein for an effective dosage is in the range of from about 100 units/ml to about 500,000 units/ml of protein, preferably in the range of from about 1000 units/ml to about 100,000 units/ml, and most preferably from about 10,000 to 100,000 units/ml. The amount of active units per ml and the duration of time of exposure depends on the nature of infection, and the amount of contact the carrier allows the lytic protein to have.

It is to be remembered that the protein works best when in a fluid environment. Hence, effectiveness of the protein is in part related to the amount of moisture trapped by the carrier. The effective amount or concentration of the active units of protein is in the range of from about 100 units/ml to about 100,000 units/ml of fluid in the wet or damp environment of the nasal and oral passages. Preferably, the protein is in the range of about 100 units/ml to about 10,000 units/ml.

Time exposure to the active protein units influences the desired concentration of active protein units per ml. It should be noted that application means that are classified as “long” or “slow” release means (such as, for example, certain nasal sprays or lozenges) provide a lower concentration of active protein units per ml, but over a longer period of time, whereas a “short” or “fast” release means (such as, for example, a gargle) provides a high concentration of active protein units per ml, but over a shorter period of time.

The therapeutic composition and method of this invention are particularly useful in mammals and specifically in humans.

Diseases that are prevented or treated with the composition and method of this invention includes, bacterial infection of burn and wounds, tuberculosis, respiratory tract infection, vaginal infection, dental infection, ulcer, digestive tract infection, ear, mouth and nose infections, eye infection, infection of mucus membrane, among others.

The invention, as described herein, discloses several methods of treatment, prophylatically or therapeutically, against variety of bacterial infections by the use of the composition of the invention. For example, respiratory tract infections are effectively treated by the method of this invention.

Two examples of bacteria which infect the upper respiratory system are Streptococcus pneumoniae and Hemophilus influenzae. In recent years, there has been an increase in the number of people, particularly children and the elderly that are infected or are carriers of penicillin resistant Streptococcus pneumoniae and Hemophilus. While these bacteria are normally harmless residents of the host, they are opportunistic organisms that are able to cause infections when the resistance of the host has been compromised. Elimination or reduction of these organisms in the upper respiratory tract will in turn reduce the occurrence or severity of diseases caused by these bacteria

According to one embodiment, the method of treatment of respiratory tract infection comprises administration of a composition containing an effective amount of at least one lytic protein produced by a bacteria being infected with a bacteriophage specific for the respiratory tract infectious bacteria and a suitable carrier for delivering the lytic protein to mouth, throat, or nasal passage. It is preferred that the lytic protein is in an environment having a pH which allows for activity of the lytic protein. If an individual has been exposed to someone with the upper respiratory disorder, the lytic protein will reside in the mucosal lining and prevent any colonization of the infecting bacteria.

The therapeutic composition and method of this invention is particularly useful in treatment or prevention of tuberculosis. Tuberculosis-associated phage proteins, for example, lytic proteins, alone or in combination with other phage proteins and other active or inactive agents, are applied by direct, or indirect application means to subjects in need thereof. Phage proteins, in the natural, modified or in a mixture of natural and modified forms, are placed in a suitable diluent or buffer and added to an appropriate carrier. If inhalers are used as carriers, the diluent can be sterile water or any water based liquid, for example. Other diluents for dispersing drugs into the bronchial tract can also be used. The examples of carriers that can be used to combat respiratory tract infections include, nasal sprays, nasal drops, nasal ointments, nasal washes, nasal injections, nasal packings, bronchial sprays, inhalers, throat lozenges, mouthwash, gargles, or ointments For the therapeutic treatment of tuberculosis, however, the use of application means such as bronchial sprays, aerosols, and inhalers are most beneficial. The phage associated lytic proteins specific for tuberculosis or Streptococcus infection were maintained in a stabilizing buffer to maintain a pH range of from about 4.0 to from about 9.0.

Bacteria-associated phage proteins or peptides and peptide fragments thereof are also useful in prophylactic and therapeutic treatment of bacterial infections of the digestive tract, including the mucus membrane. The method and composition for treating a bacterial infection of the digestive tract is similar to the methods and compositions disclosed above. Preferably, the bacterial infection of the digestive tract is caused by Gram negative bacteria selected from the group consisting of Listeria, Salmonella, E. coli, and Campylobacter. However, this method and composition effectively treat other bacteria, when the appropriate lytic protein is used.

Application means used for treating digestive tract infection includes, for example, suppository enemas, syrups, or enteric coated pills. These carriers are manufactured by conventional methods, except that in order to prevent denaturation of the protein, the protein should be incorporated into a carrier that does not contain alcohol and has been cooled to a temperature that does not cause the denaturing of the protein. Suppositories are known in the art, and are made of glycerin, fatty acids, and similar type substances that dissolve at body temperature. As the suppository dissolves, the phage proteins are released. Additionally, phage proteins could be incorporated in a lyophilized state, or may be incorporated in a liposome before being placed in the suppository, syrup or enteric coated pill.

The composition used to treat digestive tract infection has a pH of from bout 2 and about 11, more preferably a pH of from about 4.0 to about 9.0, and most preferably a pH of from about 5.5 to about 7.5. Preferably, the phage protein is in a stabilizing buffer environment prior to its addition to the carrier. The pH of the stabilizing buffer is preferably from about 4.0 to about 9.0, more preferably from about 5.5 to about 7.5 and most preferably at about 6.1. The stabilizing buffer should allow for the optimum activity of the lytic and holin proteins.

Similar compositions, as disclosed above, are used for therapeutic or prophylactic treatment of bacterial infections of bums and wounds of the skin. The carriers used to deliver therapeutic composition of the invention to the site of bums and wounds include, but are not limited to, an aqueous liquid, an alcohol base liquid, a water soluble gel, a lotion, an ointment, a non-aqueous liquid base, a mineral oil base, a blend of mineral oil and petrolatum, lanolin, liposomes, protein carriers such as serum albumin or gelatin, powdered cellulose, carmel, and combinations thereof. The composition can also be applied by a smear, spray, a time-release patch, a liquid absorbed wipe, and combinations thereof. One or more phage proteins may also be applied together to a bandage. The bandages may be sold damp or dry, wherein the protein(s) is in a lyophilized form on the bandage. This method of application is most effective for the treatment of bums.

In preferred embodiments of the invention, the lytic proteins for Pseudomonas, Staphylococcus, and Streptococcus, jointly or individually, may be incorporated into the carrier, or into a bandage to be used on bum patients, or in a solution or cream carrier.

Yet another use of lytic proteins is for the prophylactic or therapeutic treatment of vaginal infections. This treatment comprises treating the vaginal infection with an effective amount of at least one lytic protein produced by a bacteria being infected with a bacteriophage specific for that bacteria, wherein that lytic protein is incorporated in a carrier to be placed in a vagina. The lytic protein(s) used to treat bacterial infections of the vagina may be either supplemented by chimeric and/or shuffled lytic proteins, or may be itself a chimeric and/or shuffled lytic protein. Similarly, a holin protein may be included, which may also be a chimeric and/or shuffled lytic protein. The preferred carrier is a tampon, or vaginal douche. A pad may also be used as an application means, though it is not as effective. While any number of bacteria could be treated using this composition and method, it is believed that the most optimum use of this treatment composition and method would be for the treatment of an E. coli and Streptococcus B infection.

Vaginal infections caused by Group B Streptococcus can cause neonatal meningitis resulting in brain damage and premature death. Phage lytic proteins incorporated into tampon specific for group B Strep eliminates the group B organisms without disturbing normal flora so that woman would not be overcome by yeast infection post antibiotic therapy. The use of the lytic protein in the vagina would best provide a prophylactic effect, although therapeutic use would also be advisable.

To produce a pad or tampon containing the protein, the lytic protein can be applied in a solution to the tampon, and allowed to dry. The lytic protein may be incorporated into the pad or tampon by any other means known in the art, including lyophilization, spraying, etc. The tampons and pads may also be kept slightly moist, and in a sealed wrapper until ready for use. In that case, bactericide and bacteriostatic compounds and inhibitors should be present in the tampons and pads. The composition of this invention is also incorporated into a vaginal suppository. The vaginal suppository is, for example, a standard vaginal suppository, comprised of glyceride, alginate, starch, other standard binders and any combinations thereof.

When using a tampon as the application means, it is best to insert the tampon in the vagina and leave it in for up to 12 hours to distribute the protein efficiently.

As with other lytic proteins, it is preferable that the pH be kept in a range of about 4.0 and about 9.0 even more preferably at a pH range of between about 5.5 and about 7.5. As described above with the other lytic protein, the pH can be moderated by the use of a buffer. The buffer may contain a reducing agent, and more specifically dithiothreitol. The buffer may also contain a metal chelating reagent, such as ethylenediaminetetracetic disodium salt or the buffer may be a citrate-phosphate buffer. As with all compositions described in this patent, the composition may, further include a bactericidal or bacteriostatic agent as a preservative.

The lytic protein is preferably present in a concentration of about 100 to about 500,000 active protein units per milliliter of fluid in the wet environment of the vaginal tract, preferably about 100 to about 100,000 active protein units per milliliter of fluid, and preferably present in a concentration of about 100 to about 10,000 active protein units per milliliter of fluid in the wet environment of the vaginal tract.

Another use of the invention is for the prophylactic and therapeutic treatment of eye infections. The method of treatment comprises administering eye drops which comprise an effective amount of at least one lytic protein produced by the bacteria being infected with a bacteriophage specific for the bacteria and a carrier capable of being safely applied to an eye, with the carrier containing the lytic protein . In a preferred embodiment of the invention, the bacteria being treated is Hemophilus or Staphylococcus The eye drops are in the form of an isotonic solution. The pH of the solution should be adjusted so that there is no irritation of the eye, which in turn would lead to possibly infection by other organisms, and possibly to damage to the eye. While the pH range should be in the same range as for other lytic proteins, the most optimal pH will be in the range of from 6.0 to 7.5. Similarly, buffers of the sort described above for the other lytic proteins should also be used. Other antibiotics which are suitable for use in eye drops may be added to the composition containing the lytic proteins. Bactericides and bacteriostatic compounds may also be added. As stated above, this lytic protein may be either supplemented by chimeric and/or shuffled lytic proteins, or may be itself a chimeric and/or shuffled lytic protein. Similarly, a holin protein may be included, which may also be a chimeric and/or shuffled lytic protein.

It is to be remembered that all of the proteins can be used for prophylactic and therapeutic treatments of the bacteria for which the proteins are specific.

It is also to be remembered that a carrier may have more than one lytic protein. For instance, A throat lozenge may comprise just a lysin protein (which lyses the Streptococcus A strain causing “strep” throat, or it may also include the lytic proteins for Hemophilus. Similarly, the carrier for treating bums and wounds, or infections of the skin, may contain just one lytic protein, or a combination of lytic proteins, for the treatment of Pseudomonas, Streptococcus, Staphylococcus, or any other of a number of bacteria.

Lytic proteins can also be used to fight dental carries. Specifically, a lytic protein specific for Streptococcus mutans may be incorporated in a toothpaste or oral wash. Similarly, this lytic protein may also be incorporated into a chewing gum or lozenge. Any other carrier can be used that allows for the exposure of the mouth, gums, and teeth to the lytic protein.

The lytic protein may also be incorporated in a lyophilized or dried form in tooth powder. If the lytic protein is to be used in an oral wash, it is preferred that the oral wash does not contain any alcohol, so as not to denature the protein. The protein can also be in a liposome when mixed in with the toothpaste or oral wash. The concentrations of the protein units per ml of toothpaste or mouth wash can be in the range of from about 100 units/ml to about 500,000 units/ml of composition, preferably in the range of about 1000 units/ml to about 100,000 units/ml, and most preferably from about 10,000 to 100,000 units/ml. The pH of the toothpaste or oral wash should be in a range that allows for the optimum performance of the protein, while not causing any discomfort to the user of the toothpaste or oral wash. Again, as with the other uses of lytic proteins, the lytic protein use to treat dental caries may be either supplemented by chimeric and/or shuffled lytic proteins, or may be itself a chimeric and/or shuffled lytic protein. Similarly, a holin protein may be included, which may also be a chimeric and/or shuffled lytic protein.

### EXAMPLES

### Example 1

Harvesting Phage Associated Lytic Protein

Group C streptococcal strain 26RP66 (ATCC #21597) or any other group C streptococcal strain is grown in Todd Hewitt medium at 37.degree. C. to an OD of 0.23 at 650 nm in an 18 mm tube. Group C bacteriophage (C1) (ATCC #21597-B1) at a titer of 5.times.10.sup.6 is added at a ratio of 1 part phage to 4 parts cells. The mixture is allowed to remain at 37.degree. C. for 18 min at which time the infected cells are poured over ice cubes to reduce the temperature of the solution to below 15.degree. C. The infected cells are then harvested in a refrigerated centrifuge and suspended in 1/300th of the original volume in 0.1M phosphate buffer, pH 6.1 containing 5.times.10.sup.-3 M dithiothreitol and 10 ug of DNAase. The cells will lyse releasing phage and the lysin protein. After centrifugation at 100,000.times. g for 5 hrs to remove most of the cell debris and phage, the protein solution is aliquoted and tested for its ability to lyse Group A Streptococci.

The number of units/ml in a lot of protein is determined to be the reciprocal of the highest dilution of protein required to reduce the OD650 of a suspension of group A streptococci at an OD of 0.3 to 0.15 in 15 minutes. In a typical preparation of protein 4.times.10.sup.5 to 4.times.10.sup.6 units are produced in a single 12 liter batch.

Use of the protein in an immunodiagnostic assay requires a minimum number of units of lysin protein per test depending on the incubation times required. The protein is diluted in a stabilizing buffer maintaining the appropriate conditions for stability and maximum enzymatic activity, inhibiting nonspecific reactions, and in some configurations contains specific antibodies to the Group A carbohydrate. The preferred embodiment is to use a lyophilized reagent which can be reconstituted with water. The stabilizing buffer can comprise a reducing reagent, which can be dithiothreitol in a concentration from 0.001M to 1.0M, preferably 0.005M. The stabilizing buffer can comprise an immunoglobulin or immunoglobulin fragments in a concentration of 0.001 percent to 10 percent, preferably 0.1 percent. The stabilizing buffer can comprise a citrate-phosphate buffer in a concentration from 0.001M to 1.0M, preferably 0.05M. The stabilizing buffer can have a pH value in the range from 5.0 to 9.0. The stabilizing buffer can comprise a bactericidal or bacteriostatic reagent as a preservative. Such preservative can be sodium azide in a concentration from 0.001 percent to 0.1 percent, preferably 0.02 percent.

The preparation of phage stocks for lysin production is the same procedure described above for the infection of group C streptococcus by phage in the preparation of the lysin protein. However, instead of pouring the infected cells over ice, the incubation at 37.degree. C. is continued for a total of 1 hour to allow lysis and release of the phage and the protein in the total volume. In order for the phage to be used for subsequent lysin production the residual protein must be inactivated or removed to prevent lysis from without of the group C cells rather than phage infection.

The use of chimeric or shuffled proteins shows a great improvement as to the properties of the protein, as illustrated by the following examples:

### Example 2

Production of Chimeric Lytic Proteins

A number of chimeric lytic proteins have been produced and studied. Gene E-L, a chimeric lysis constructed from bacteriophages phi X174 and MS2 lysis proteins E and L, respectively, was subjected to internal deletions to create a series of new E-L clones with altered lysis or killing properties. The lytic activities of the parental genes E, L, E-L, and the internal truncated forms of E-L were investigated in this study to characterize the different lysis mechanism, based on differences in the architecture of the different membranes spanning domains. Electron microscopy and release of marker proteins for the cytoplasmic and periplasmic spaces revealed that two different lysis mechanisms can be distinguished depending on penetrating of the proteins of either the inner membrane or the inner and outer membranes of the E. coli. FEMS Microbiol. Lett. Jul. 1, 1998 164(1); 159-67.

Also, an active chimeric cell wall lytic protein (TSL) is constructed by fusing the region coding for the N-terminal half of the lactococcal phage Tuc2009 lysin and the region coding for the C-terminal domain of the major pneumococcal autolysin. The chimeric protein exhibited a glycosidase activity capable of hydrolysing choline-containing pneumoccal cell walls.

### Example 3

Isolation of the Pal Lytic Protein

Recombinant E. coli DH5 (pMSP11) containing the pal lytic protein gene were grown overnight, induced with lactose, pelleted, resupended in phosphate buffer, broken by sonication. After centrifugation, the Pal protein in the supernatant was purified in a single step using a DEAE-cellulose column and elution with choline. Protein content was analyzed with the Bradford method. Using this method, a single protein band was identified by SDS-PAGE.

### Example 4

Killing Assay

S. pneumoniae of various serotypes and 8 different viridans streptococi were grown overnight and for most assays diluted and re-grown for 6 h to log phase of growth, pelleted and resupended in 0.9% saline to an OD @ 620 nm of 1.0. In some experiments, stationary phase organisms were used. Killing assays were performed by adding 100, 1,000 or 10,000 U/mL of Pal to an equal volume of the bacterial suspension and incubating for 15 minutes at 37 C. Phosphate buffer served as control in place of protein. Bacterial counts before and after Pal or control phosphate buffer treatment were assessed by serial 10-fold dilutions at various time points and plated to determine colony forming units.

One unit (U) of Pal was defined as the highest dilution at which Pal decreased the OD of a pneumococcal strain by half in 15 minutes.

### Example 5

Susceptability of Oral Streptoccocci to Pal Protein

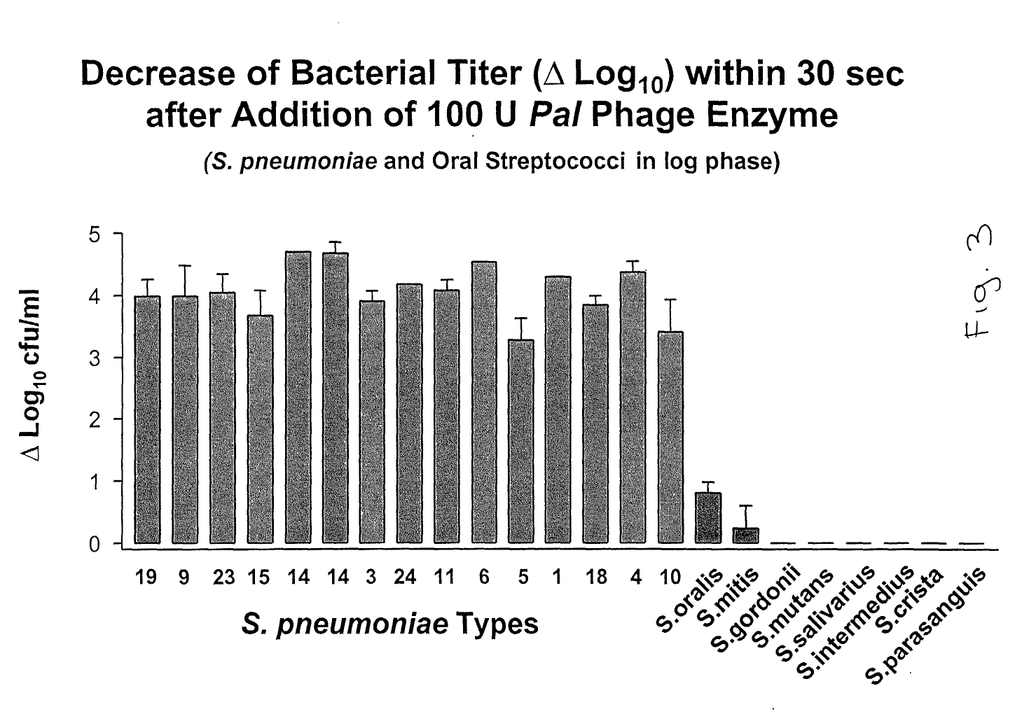

Various serotypes of oral streptoccoci were tested against bacteria-associated lytic proteins, in particular, the Pal protein. A variety of S. pneumoniae type bacteria was also included in the test. Pal protein were used at a concentration of 100 U of the purified protein. As can be seen in FIG. 3 all S. pneumoniae serotypes are killed (˜4 logs) within the 30 seconds of exposure. Of the oral streptococci tested, only S. oralis and S. mitis show low sensitivity to the Pal protein. Tables II & shows the number of bacteria dramatically decreases after the addition of lysin and this decrease has a direct relationship with the dose of proteins used Table III confirms similar results in vivo

TABLE I
Bacterial Strains Tested for Lysin Sensitivity

| Bacteria | Stran | Comment | Source |
|---|---|---|---|
| Set I. Group A Streptococci | | | |
| Group A Streptococcus | J17A4 | Grouping state | 1 |
| Group A Streptococcus | JRS75 | No M protein | 1 |
| Group A Streptococcus | D710 | Class I (M1) | 1 |
| Group A Streptococcus | D471 | Class I (M6) | 1 |
| Group A Streptococcus | A374 | Class I (M12) | 1 |
| Group A Streptococcus | IRP43 | Class I (M19) | 1 |
| Group A Streptococcus | IRP256 | Class II (M2) | 1 |
| Group A Streptococcus | 0691 | Class II (M11) | 1 |
| Group A Streptococcus | D734 | Class II (M22) | 1 |
| Group A Streptococcus | A945 | Class II (M49) | 1 |
| Group A Streptococcus | A486 variant | No A | 1 |
| | | carbohydrate | |
| Set II. Other Lancefield Groups | | | |
| Group B Streptococcus | D908 | | 1 |
| Group C Streptococcus | 26RP66 | | 1 |
| Group D Streptococcus | D76 | | 1 |
| Group E Streptococcus | K131 | | 1 |
| Group F Streptococcus | F68D | | 1 |
| Group G Streptococcus | D166B | | 1 |
| Group L Streptococcus | D167A | | 1 |
| Group N Streptococcus | C559 | | 1 |
| Set III. Oral Streptococci | | | |
| Streptococcus crista | PK1408 | AKA CC$A | 2 |
| | | strep. | |
| Streptococcus intermedius | PK2821 | | 2 |
| Streptococcus gordonnri | FSS2 | | 3 |
| Streptococcus gurdonn | DLt | | 2 |
| Streptococcus gordonii | PK488 | | 2 |
| Streptococcus gordonnri | PK2565 | Blackburn | 2 |
| | | stran | |
| Streptococcus millis | J22 | | 2 |
| Streptococcus mutans | NG5 | | 4 |
| Streptococcus mutans | Ingbritt 175 | | 4 |
| Streptococcus oralis | Ht | | 2 |
| Streptococcus oralis | PK34 | | 2 |
| Streptococcus parasanguis | PK2564 | | 2 |
| Streptococcus salivanus | ATCC 9222 | | 2 |
| Streptococcus salivanus | ATCC 7945 | | 2 |
| Set IV. Non-strep Bacteria | | | |

TABLE II
In vitro Killing of Group A Streptococci by Lysin

| Lysin | Starting | | | | | |
|---|---|---|---|---|---|---|
| Units | count | 5 sec | 30 sec | 60 sec | 5 min | 10 min |
| 1000 | 5 × 10⁶ | 0 | 0 | 0 | 0 | 0 |
| 100 | 8.6 × 10⁶ | 1530 | 1196 | 771 | 64 | 6 |
| 10 | 9.8 × 10⁶ | >3000 | >3000 | >3000 | >3000 | >3000 |

TABLE III
Mouse Colonization by Lysin (1000 U)
Treated Group A Streptococci

| | Mouse | Day 1 | Day 2 | Day 3 | Day 7 |
|---|---|---|---|---|---|
| Lysin | 1 | 0 | 0 | 0 | 0 |
| Treated | 2 | 0 | 0 | 0 | 0 |
| | 3 | 0 | 0 | 0 | 0 |
| | 4 | 0 | 0 | 0 | 0 |
| | 5 | 0 | 0 | 1 | 0 |
| | Total | 0/5 | 0/5 | 1/5 | 0/5 |
| | Mouse | Day 1 | Day 2 | Day 3 | Day 7 |
| Buffer | 1 | 26 | 14 | 7 | 0 |
| Treated | 2 | >300 | 17 | 100 | 83 |
| | 3 | 9 | 0 | 15 | 0 |
| | 4 | >300 | >300 | >300 | 220 |
| | 5 | 2 | 2 | 30 | 0 |
| | Total | 5/5 | 4/5 | 5/5 | 2/5 |

### Example 6

Susceptability of Stationary Phase Bacteria to Lytic Protein

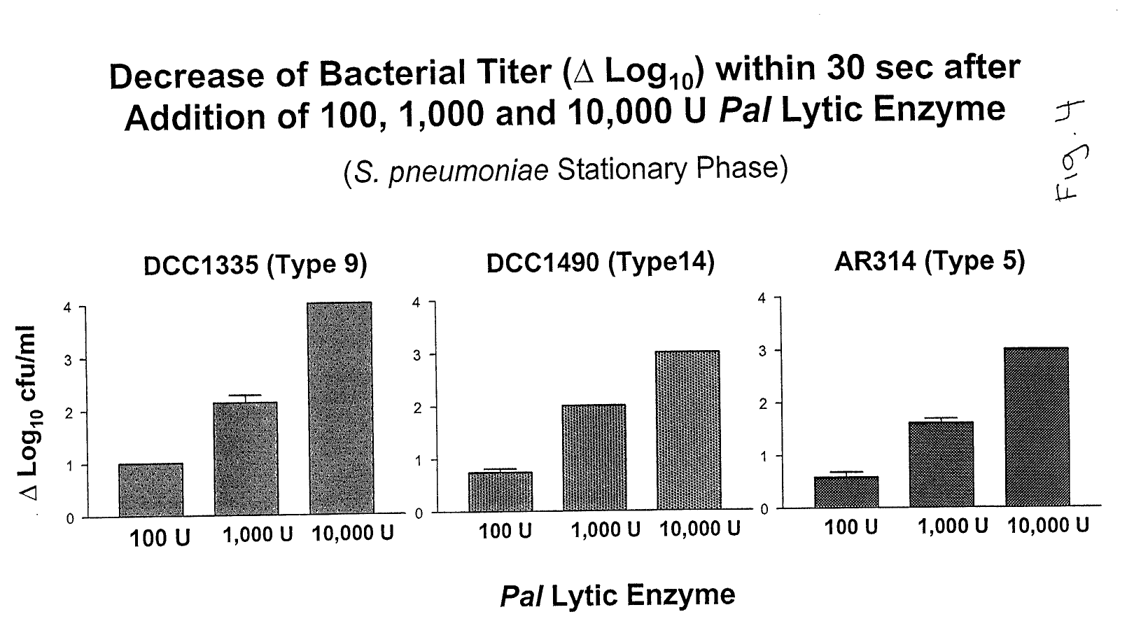

In order to confirm that activity of lytic proteins are independent of the bacterial growth, several serotypes of serotypes of S.pneumoniae at stationary phase of growth were tested against lytic proteins. In particular, 3 strains of Pal lytic protein were used against 3 sereotypes of S. pneumoniae. The results show that all bacterial strains tested against Pal protein were killed in 30 seconds (see FIG. 4). An approximately 2-log drop in viability of the bacteria occurred with 1,000 U of protein, as opposed to about 3-4 log drop in the viability with 10,000 units.

In vivo results are in animals confirm these results (See Tables, IV and V)

TABLE IV
Pretreatment of Mice with Lysin (250 U)
Prevents Streptococcal Infections

| | | Lysin | | | Control | | |
|---|---|---|---|---|---|---|---|
| | | Mouse | Day 1 | Day 2 | Mouse | Day 1 | Day 2 |
| | | 1 | 0 | 0 | 1 | 1 | 14 |
| | | 2 | 0 | 0 | 2 | 33 | 250 |
| | | 3 | 9 | 0 | 3 | 0 | 0 |
| | | 4 | >300 | >300 | 4 | 0 | 0 |
| | | 5 | 1 | 0 | 5 | 4 | 12 |
| Crude | {open oversize parenthesis} | 6 | 0 | 0 | 6 | 1 | 2 |
| | | 7 | 0 | 0 | 7 | 6 | >300 |
| | | 8 | 0 | 0 | 8 | 6 | 0 |
| | | 9 | 0 | 0 | 9 | >300 | Dead |
| | | 10 | 0 | 0 | 10 | 83 | >300 |
| | | 11 | 0 | 0 | 11 | 10 | >300 |
| | | 12 | 1 | 0 | 12 | 0 | 0 |
| | | 13 | 0 | 0 | 13 | 0 | nd. |
| | | 14 | 1 | 1* | 14 | 150 | nd. |
| | | 15 | 11 | 10* | 15 | 0 | nd. |
| Purified | {open oversize parenthesis} | 16 | 0 | 0 | 16 | >300 | nd. |
| | | 17 | 0 | nd. | 17 | 200 | nd. |
| | | 18 | 0 | nd. | Total | 12/17 | 8/12 |
| | | 19 | 0 | nd. | | | |
| | | 20 | 0 | nd. | | | |
| | | 21 | 0 | nd. | | | |
| | | Total | 6/21 | 3/16 | | | |

nd. no data collected
*isolated streptococci remained sensitive to lysin treatment in vito

TABLE V
Elimination of Group A Streptococci from the
Mucosal Surface of Colonized Mice (500 U lysin/mouse)

| 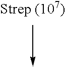 | | | | 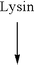 | | | |
|---|---|---|---|---|---|---|---|
| | CFUs | | | | (post lysin treatment) | | |
| | Day | Day | Day | Day | (2 hr) | (24 hr) | (45 hr) |
| Mouse | 1 | 2 | 3 | 4 | Day 4 | Day 5 | Day 6 |
| 1 | >300 | >300 | >300 | >300 | 0 | 0 | 200* |
| 2 | >300 | >300 | >300 | >300 | 0 | 50* | 0 |
| 3 | >300 | >300 | >300 | >300 | 0 | 0 | 0 |
| 4 | >300 | >300 | >300 | >300 | 0 | 0 | 0 |
| 5 | >300 | >300 | >300 | >300 | 0 | Dead | Dead |
| 6 | >300 | >300 | >300 | >300 | 0 | nd | 0 |
| 7 | >300 | >300 | >300 | >300 | 0 | nd | 0 |
| 8 | >300 | >300 | >300 | >300 | 0 | nd | 0 |
| 9 | >300 | >300 | >300 | >300 | 0 | nd | 0 |
| Total | 9/9 | 9/9 | 9/9 | 9/9 | 0/9 | 2/5 | 2/9 |
| Colonized | | | | | | | |

nd, no sedate collected
*isolated streptococci remained sensitive to lysin treatment in vitro

### Example 7

Effect of Pal Lytic Protein on Log-Phase and Stationary Phase Oral Streptococci

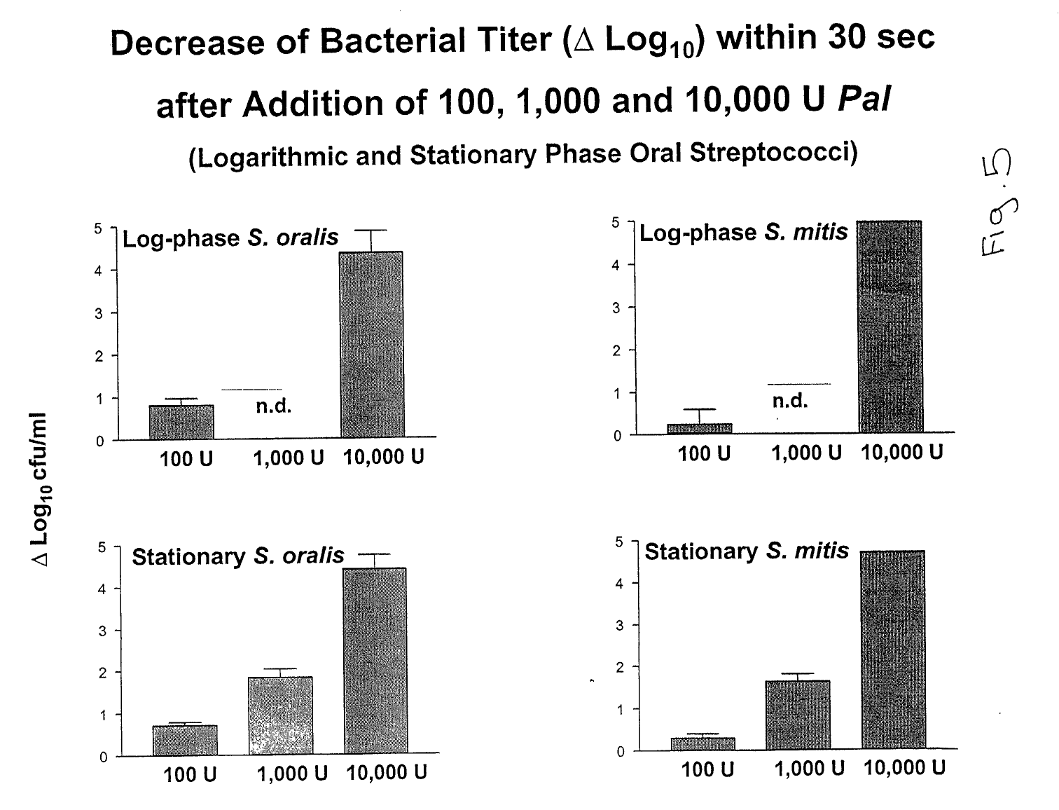

Streptococci oralis and Streptococci.mitis in log or stationary phases of growth were treated with different concentrations of the Pal lytic protein. Viability was measured after 30 seconds. Results, as shown in FIG. 5, indicate that both bacterial species were equally sensitive to the Pal protein in both log or stationary phases of growth.

Equivalents

Those skilled in the art will recognize, or be able to ascertain using no more than routine experimentation, many equivalents to the specific embodiments of the invention described herein. Such equivalents are intended to be encompassed by the following claims.

Each publication cited herein is incorporated by reference in its entirety.

All publications and applications cited in this disclosure are specifically incorporated by reference in their entireties. The contents of priority documents U.S. application Ser. No. 09/497,495 filed Apr. 14, 2000; Ser. No. 09/395,636 filed Sep. 14, 1999 and Ser. No. 08/962,523 filed Oct. 31, 1997 specifically are incorporated in their entireties by reference. Related U.S. patent application Ser. No. 09/482,992 filed Jan. 14, 2000; Ser. No. 09/497,495 filed Apr. 14, 2000; Ser. No. 09/654,483 filed Sep. 1, 2000; Ser. No. 09/653,690 filed Sep. 1, 2000; Ser. No. 09/671,882 filed Sep. 28, 2000; Ser. No. 09/671,881 filed Sep. 28, 2000; Ser. No. 09/671,880 filed Sep. 28, 2000; Ser. No. 09/671,879 filed Sep. 28, 2000; Ser. No. 09/671,878 filed Sep. 28, 2000; Ser. No. 09/671,991 filed Sep. 28, 2000; Ser. No. 09/671,992 filed Sep. 28, 2000; Ser. No. 09/671,990 filed Sep. 28, 2000; Ser. No. 09/560,650 filed Apr. 28, 2000 and Ser. No. 09/704,148 filed Nov. 2, 2000 are incorporated by reference in their entireties.

It is to be understood that the description, specific descriptions of embodiments and examples, while indicating exemplary embodiments, are given by way of illustration and are not intended to limit the present invention. Various changes and modifications within the present invention will become apparent to the skilled artisan from the discussion, disclosure and data contained herein, and thus are considered part of the invention.

What we claim is:

1. A pharmaceutical composition comprising an effective amount of at least one phage lytic and/or holin protein, or peptides , and a pharmaceutically acceptable carrier.

2. The pharmaceutical composition according to claim 1, wherein said lytic and holin protein or peptides s are derived from the same or different bacteria.

3. The pharmaceutical composition according to claim 1, wherein said lytic and holin protein or peptides are derived from the same or different bacteriophages.

4. The pharmaceutical composition according to claim 1, wherein said protein or peptides are natural, modified, or a combination thereof.

5. The pharmaceutical composition according to claim 4, wherein said modified protein or peptides are produced by chemical synthesis, DNA recombination technique, or both.

6. The pharmaceutical composition according to claim 4, wherein said protein or peptides are produced by chimerization, shuffling, or both.

7. The pharmaceutical composition according to claim 1, wherein said carrier comprises agents suitable for delivering said protein or peptides to the site of the infection.

8. The pharmaceutical composition according to claim 1, wherein said protein or peptides comprises, an antibody or an antibody fragment, having biological activity either alone or with combination of other peptide molecules.

9. The pharmaceutical composition according to claim 1, wherein at least one phage lytic or holin protein is produced by infection of bacteria with bacteriophage wherein the bacteria is selected from the group consisting of Streptococci, Pseudomonas, Pneumococci, Salmonella, Staphylococci, Shigella, Haemophilus, Listeria, Mycobacteria, Vibrio, Corynebacteria, Bacillus, Spirochete, Myxococcus, Burkholderia, Brucella, Yersinia, Clostridium, Campylobacter, Neisseria, Actinomycetes, Agrobacterium, Alcaligenes, Clostridium, Coryneforms, Cyanobacteria, Enterobacteria, Lactobacillus, Lactoctococcus, Micrococcus, Pasteurella, Rhizobium, Xanthomonas, Bdellovibrio, mollicutes, Chlamydia, Spiroplasma, Caulobacter, Aeromonas, Bdellovibrio, Caulobacter, Chlamydia, Clostridium, Coliform, Coryneforms, Listeria, Micrococcus, Mycobacterium, Lacticola, Pasteurella, or a combination thereof.

10. The pharmaceutical composition according to claim 9, wherein said bacteriophage is selected from the group consisting of A1-Dat, Bir, M1, MSP8, Pal, R1, R2, SV2,VP5, PhiC, φ31C, φUW21, φ115-A, φ150A, 119, SK1, 108/016, 29, 37, 43, 51, 59.1, PM2, AP50, φNS11, BLE, Ipy-1, MP15, mor1, BP1, SPP1, Spbb, type F, alpha, φ105, 1A, II, Spy-2, SST, G, MP13, PBS1, SP3, SP8, SP10, SP15, SP50, MAC-1, MAC-1′, MAC-2, MAC-4, MAC-4′, MAC-5, MAC-7, φCb2, φCb4, φCb5, Cb8r, φCb9, φCB12r, φCb23r, φCP2, φCP18, φCr14, φCr28, PP7, φCb2, φCb4, φCb5, φCb8r, Cb9, φCB12r, φCb23r, φCP2, φCP18, φCr14, φCr28, PP7, Chp-1, F1, HM7, HM3, CEB, AE2, A, Ec9, f1, fd, HR, M13, ZG/2, ZJ/2, Arp, BL3, CONX, MT, Beta, A8010, A19, S-2L, S-4L,AS-1, S-6(L), C-2, If1, If2, Ike, I2-2, PR64FS, SF, tf-1, PRD1, H-19J, B6, B7, C-1,C2, Jersey, G/3A, T5, ViII, b4, chi, Beccles, tu, PRR1, 7s, C-1, c2, fcan, folac, Ialpha, M, pilhalpha, R23, 34, ZG/1, ZIK/1, ZJ/1, ZL/3, ZS/3, alpha15, f2, fr, FC3-9, K19, Mu, 01, P2, ViI, φ92, 121, 16-9, 266, C16, DdVI, PST, SMB, SMP2, a1, 3, 3T+, 9/0, 11F, 50, 66F, 5845, 8893, M11, QB, ST, W18, VK, FI, ID2, fr, f2, H387, 2389, 2671, 2685, 4211, N1, N5, Lacticola, Leo, R1-Myb, 13, 2, 32, AU, Phi6, Pf1, Pf2, Pf3, D3, Kf1, M6, PS4, SD1, PB-1, PP8, PS17, nKZ, nW-14, n1, 12S, 3A, B11-M15, 77, 107, 187, 2848A, Twort, A25, A25 PE1, A25 VD13, A25 omega8, A25 24, OXN-52P, VP-3, VP5, VP11, alpha3alpha, IV, kappa, 06N-22-P, VP1, x29, II, nt-1, Cf, Cflt, f, Xf2, XP5, or a combination thereof.

11. The pharmaceutical composition according to claim 8, wherein said phage protein or peptide comprises natural protein or peptide, a naturally occurring allelic variant , isozyme or analogue , of said protein or peptide, a modified protein or peptide which is encoded by a nucleic acid molecule comprising a nucleotide sequence which is at least 65% identical to a nucleic acid encoding the said natural protein or peptide.

12. The pharmaceutical composition according to claim 1, wherein said composition is used for therapeutic or prophylatic treatment of an infection caused by a bacterium selected from the group consisting of Hemophilus influenza, Pseudomonas, Streptococcus pneumoniae, Streptococcus fasciae, Listeria, Salmonella, E. coli, Campylobacter, Helicobacter pylori, Pseudomonas. Streptococcus mutans, Mycobacterium tuberculosis and Streptococcus, or a combination thereof.

13. The pharmaceutical composition according to claim 8 wherein said protein or peptides further comprising heterologous amino acid sequences

14. The pharmaceutical composition according to claim 8 wherein said antibody selectively binds to said phage protein or protein peptides fragments.

15. The pharmaceutical composition according to claim 8 wherein said nucleic acid molecules are attached to one or more regulatory sequences and signal sequences, wherein said sequences affect site specificity and trans-membrane movements of said nucleic acid molecule

16. The pharmaceutical composition according to claim 1 wherein said phage protein or peptides and peptide fragments thereof are attached to a signal sequence that assists transportation of said composition to mucous membrane.

17. The pharmaceutical composition according to claim 1 wherein said composition further comprises at least one complementary agent.

18. The pharmaceutical composition according to claim 1 wherein said complementary agent is selected among the group consisting of antimicrobial agents, anti-inflammatory agents, antiviral agents, local anesthetic agents, corticosteroids, destructive therapy agents, antifungals, antiandrogens, or a combination thereof.

19. A method for treating, preventing or ameliorating bacterial infections at a mucosal surface comprising the steps:
a) obtaining a composition comprising an effective amount of at least one lytic protein peptides or peptide fragments thereof; and
b) applying said composition to the mucosal surface,
wherein the lytic protein, peptides, or peptide fragments thereof is produced by infecting a bacterium causing said infection with a bacteriophage specific for said bacteria and wherein said bacteria produces said at least one recombinant lytic protein selected from the group consisting of chimeric lytic proteins, shuffled lytic proteins, and combinations thereof.

20. The method according to claim 19 wherein said bacteria infection is selected among the group consisting of Hemophilus influenza, Pseudomonas, Streptococcus pneumoniae, Streptococcus fasciae, Listeria, Salmonella, E. coli, Campylobacter, Helicobacter pylori, Pseudomonas. Streptococcus mutans, Mycobacterium tuberculosis and Streptococcus, or a combination thereof

21. A method for detecting the presence of the phage protein, or peptides of claim 1 in a sample, comprising:
a) contacting the sample with a compound that selectively binds to said phage protein, or peptides, of claim 1; and
b) determining whether the compound binds to said phage protein or peptides in said sample.

22. The method according to claim 21, wherein the compound is an antibody or antibody fragment

23. A kit comprising a compound which selectively binds to a phage protein, or peptides of claim 1 and an instruction manual.

24. A method for detecting the presence of a gene or a gene fragment encoding a lytic protein or a peptide in a sample, comprising the steps of:
a) contacting the sample with a nucleic acid probe or primer which selectively hybridizes to gene or gene fragment ; and
b) determining whether the nucleic acid probe or primer binds to the gene or gene fragment in the sample.

25. A kit comprising a compound which selectively hybridizes to gene or gene fragment encoding a lytic protein, or peptide and instructions for use.

26. A method for identifying a compound which binds to a polypeptide of claim 8 comprising the steps of:
a) contacting a polypeptide, or a cell expressing a polypeptide of claim 8 with a test compound; and
b) determining whether the polypeptide binds to the test compound.

27. A method for modulating the activity of a polypeptide of claim 8 comprising contacting a polypeptide or a cell expressing a polypeptide of claim 8 with a compound which binds to the polypeptide in a sufficient concentration to increase or decrease of the polypeptide.

28. A method for identifying a compound that modulates the activity of a polypeptide of claim 8, comprising:
a) contacting the a polypeptide of claim 8 with a test compound; and
b) detecting an increase or decrease in the activity of the polypeptide of step a).
